(19) United States
(12) Patent Application Publication
(10) Pub. No.: US 2023/0380839 A1
(43) Pub. Date: Nov. 30, 2023

(54) ARTERIOVENOUS FISTULA BANDING DEVICE

(71) Applicant: OHIO STATE INNOVATION FOUNDATION, Columbus, OH (US)

(72) Inventors: Khaled Boubes, Dublin, OH (US); Ryan Brune, Columbus, OH (US); Corinne Macnichol, Columbus, OH (US); John Bockbrader, Columbus, OH (US)

(21) Appl. No.: 18/202,569

(22) Filed: May 26, 2023

Related U.S. Application Data
(60) Provisional application No. 63/346,086, filed on May 26, 2022.

(51) Int. Cl. A61B 17/12; A61M 1/36; A61B 34/00

(52) U.S. Cl. CPC A61B 17/12009; A61M 1/3655; A61B 2017/00876; A61M 2205/3334; A61B 34/73

(57) ABSTRACT

A arteriovenous banding device may include a band comprising a first end and a second end, the band extending circumferentially around a central axis to define a central channel having a first diameter, a housing coupled to the first end of the band and defining a housing slot to accept the second end of the band, wherein the housing is disposed outside of the central channel, a screw disposed within the housing and comprising at least one thread disposed adjacent to or abutting the band; and an actuator coupled to the screw, wherein the actuator causes rotation of the screw which causes the second end of the band to move away from or towards the first end of the band such that the first diameter of the central channel changes.

### CROSS-REFERENCE TO RELATED APPLICATIONS

This application is a continuation of U.S. Patent Application No. 63/346,086, filed May 26, 2022, which is incorporated herein by reference in its entirety.

### BACKGROUND

The present disclosure relates generally to arteriovenous fistulas (AVFs). Patients with end-stage renal disease undergoing hemodialysis (HD) often require a functional vascular access, which may include arteriovenous fistula (AVF), arteriovenous graft, or central venous catheter. AVFs are one example access type which provide for superior longevity and lower infection rate, resulting in improved patient survival and lower cost when compared to other types of access. Despite these attributes, more than a third of AVFs fail to mature. Limited blood flow at the time of fistula creation is one cause for AVF maturation failure.

Arteriovenous fistulas are made by connecting a vein to an artery. An AVF may have, for example, at least 600 cc/min of blood flow, and may be >6 mm wide. When the AVF is created, the flow within the vein immediately increases from ˜20-60 cc/min to ˜200 cc/min since it is a low-resistance circuit. In one scenario, the flow will gradually increase over the following few weeks to achieve >600 cc/min. However, because of this sudden initial increase in blood flow, and the turbulence created by it, the vein's wall usually adapts by trying to become thicker (neo-intimal hyperplasia). This will prevent the blood flow from increasing further in the AVF, which limits its use. Current solutions to fix this include repeated angioplasty (stretching with a balloon from the inside) of the AVF, which is a costly procedure with its own risks.

Surgical creation of AVFs with higher blood flow might help with increasing the maturation rate. However, surgeons are usually reluctant to create such AVFs because high uncontrollable blood flow rates may develop and result in complications such as, but not limited to, steal-syndrome, heart failure (HF), pulmonary hypertension and cephalic arch stenosis (CAS). Therefore, a need exists for arteriovenous fistulas which provide adequate blood flow while solving the problems associated with higher blood flow.

### SUMMARY

Provided herein are systems, methods, and devices for arteriovenous fistulas which can adjust the flow rate of blood through the fistula. By adjusting the diameter of the arteriovenous fistula, blood flow through the fistula is controlled which can reduce complications associated with high blood flow. For example, a device of this disclosure may be opened or expanded during hemodialysis so that blood flow is adequate to filter waste and complete the renal replacement therapy. An example device may be closed or contracted when hemodialysis is complete so that everyday blood flow does not reach levels associated with complications.

In some implementations, an arteriovenous fistula banding device is disclosed, the device including: a band including a first end and a second end, the band extending circumferentially around a central axis to define a central channel having a first diameter; a housing coupled to the first end of the band and defining a housing slot to accept the second end of the band, wherein the housing is disposed outside of the central channel; a screw disposed within the housing and including at least one thread disposed adjacent to or abutting the band; and an actuator coupled to the screw, wherein the actuator causes rotation of the screw which causes the second end of the band to move away from or towards the first end of the band such that the first diameter of the central channel changes.

In some implementations, a device, wherein the actuator is a magnet rotatably disposed within the housing. The magnet may be a diametrically magnetized magnet (e.g., a neodymium magnet).

In some implementations, a device, wherein the central channel is sized to surround an arteriovenous fistula, and wherein rotation of the screw to change the first diameter of the central channel changes an internal diameter of the arteriovenous fistula. In some implementations, the arteriovenous fistula is disposed within the central channel and the blood flow through the fistula is controllably manipulable by the device.

The device may include one or more of a variety of engageable structures to expand or contract the band around the arteriovenous fistula. Roller wheels, a pre-tensioned spring, or a spool may be used in some examples. In other implementations, a worm screw mechanism is used to engage with the band. In some implementations, the band further includes a plurality of teeth protruding from an outer side of the band, wherein the plurality of teeth is engageable with the at least one thread of the screw. In some implementations, the band further includes a plurality of slots extending through the band from an outer side to an inner side, wherein the plurality of slots is engageable with the at least one thread of the screw.

In some implementations, the flow through the arteriovenous fistula is monitored (e.g., during a dialysis procedure). A variety of standard flow sensors may be provided to gather flow data and transmit it to a control system or nearby device. In some implementations, a device is disclosed further including a flow sensor disposed adjacent to the central channel, the flow sensor configured to measure fluid flow through the central channel.

The size of the device and band can affect the pressure placed on the arteriovenous fistula as well as the flow therethrough. Additionally, the size and materials used contribute to the flexibility and possible geometry of the device. In some implementations, a device is disclosed, wherein the first diameter of the central channel is adjustable from 3 mm to 15 mm. In some implementations, a device, wherein a width of the band as measured in a direction parallel to the central axis is in a range from 5 mm to 10 mm. In some implementations, a device, wherein the device includes a flexible resin and wherein the device is 3D printed in a curved configuration such that the band is biased towards the curved configuration.

In some implementations, a device, wherein the housing further includes a magnet cover piece separably couplable to the housing to cover the magnet.

A mechanism for attaching the device within a patient is disclosed, wherein a surgeon may attach the device adjacent to the arteriovenous fistula. In some implementations, a device, further including a suture protrusion extending out from the housing in a direction transverse to the band, wherein the suture protrusion is disposed outside of the central channel.

Containing the device to a desired location is disclosed, including method of preventing unnecessary movement or implantation failure of the device. In some implementations, a device, wherein the housing further includes an outer band housing enveloping the band and the central channel wherein the second end of the band is contained within the outer band housing regardless of the first diameter of the central channel. In some implementations, a device, wherein the outer band housing includes a track engageable with a portion of the band such that the band follows the track as it extends or retracts to expand or contract the central channel. In some implementations, a device, wherein the second end of the band further includes a mechanical stop to define a maximum diameter of the central channel. In some implementations, a device, wherein the plurality of teeth extend only from a distal portion of the band such that a proximal portion of the band defines a minimum diameter of the central channel.

A motor may also be used as the actuator such that the device may include internal driving of the screw to expand or contract the band. In some implementations, a device, wherein the actuator is a motor with a shaft coupled to the screw.

In some implementations, a device, further including: a control system including: a flow sensor disposed adjacent to the central channel, the flow sensor configured to measure fluid flow through the central channel; and a controller configured to receive a measured fluid flow, compare the measured fluid flow to a predetermined fluid flow, and drive the actuator to adjust the first diameter of the central channel based on the predetermined fluid flow.

In other implementations, a system is disclosed, the system including: an arteriovenous fistula banding device, the device including: a band including a first end and a second end, the band extending circumferentially around a central axis to define a central channel having a first diameter; a housing coupled to the first end of the band and defining a housing slot to accept the second end of the band, wherein the housing is disposed outside of the central channel; a screw disposed within the housing and including at least one thread disposed adjacent to or abutting the band; and a magnet rotatably disposed within the housing coupled to the screw, wherein rotation of the screw causes the second end of the band to move away from or towards the first end of the band such that the first diameter of the central channel changes; and an external driver including: a driver magnet; and a handle coupled to the driver magnet wherein rotating the handle of the external driver rotates the driver magnet about a turning axis and causes the magnet of the arteriovenous fistula banding device to rotate.

To drive the magnet of the device, an external electromagnetic field may be applied by a mechanical or electromechanical device. In some implementations, a system is disclosed, wherein the arteriovenous fistula banding device is disposed internal to a patient such that an arteriovenous fistula is disposed inside the central channel, and wherein the external driver is disposed outside of the patient such that the driver magnet of the external driver aligns with the magnet of the arteriovenous fistula banding device.

In some implementations, a system, further including: a control system including: a flow sensor disposed adjacent to the central channel, the flow sensor configured to measure fluid flow through the central channel; and a controller configured to receive a measured fluid flow from the flow sensor, compare the measured fluid flow to a predetermined fluid flow, and drive the external driver to adjust the first diameter of the central channel to a second diameter based on the predetermined fluid flow. In some implementations, a system, wherein the control system forms a feedback loop to continuously adjust the first diameter of the central channel. In some implementations, a system, wherein the predetermined fluid flow may be changed by a remote controller, which is either the same as or different from the controller of the control system, based on a type of procedure to be performed. In some implementations, a system, wherein the first diameter of the central channel limits the fluid flow and a second diameter larger than the first diameter increases fluid flow during a hemodialysis procedure.

In other implementations, a method of controlling blood flow through an arteriovenous fistula is disclosed, the method including: providing an arteriovenous fistula banding device including a band extending circumferentially to define a central channel having a first diameter, a screw having at least one thread disposed adjacent to or abutting the band, and a magnet coupled to the screw, wherein the arteriovenous fistula is disposed within the central channel; rotating the magnet to cause rotation of the screw such that the at least one thread of the screw contacts a portion of the band to expand or contract the central channel to a second diameter; and expanding or contracting an inner diameter of the arteriovenous fistula such that a fluid flow rate through the arteriovenous fistula is increased or decreased.

In some implementations, a method, further including: measuring the fluid flow rate through the central channel via a flow sensor disposed adjacent to the central channel.

In some implementations, a method, further including: receiving, at a controller, a measured fluid flow rate; comparing the measured fluid flow rate to a predetermined fluid flow rate; and adjusting the first diameter of the central channel to a second diameter based on a difference between the measured fluid flow rate and the predetermined fluid flow rate.

In some implementations, a method, further including: expanding the first diameter of the central channel and the inner diameter of the arteriovenous fistula for a duration of a hemodialysis process; and contracting the first diameter of the central channel and the inner diameter of the arteriovenous fistula when the hemodialysis process is complete.

In some implementations, a method, wherein rotating the magnet and the screw is accomplished by an external driver having a driver magnet, the external driver disposed outside a patient's body and adjacent to the arteriovenous fistula banding device.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 provides an illustration of a surgical creation of an arteriovenous fistula, according to one implementation.

FIG. 2 illustrates an example device implanted in a patient at a desired point on an arteriovenous fistula, according to one implementation.

FIG. 3 shows a perspective view of an arteriovenous fistula banding device, according to one implementation.

FIG. 4 shows a front view of the device of FIG. 3.

FIG. 5 shows and exploded view of the device of FIGS. 3 and 4, according to one implementation.

FIG. 6 shows a perspective view of the assembled device of FIG. 5.

FIG. 7 shows a side view of the assembled device of FIG. 5.

FIG. 8 shows a back view assembled device of FIG. 5.

FIG. 9 shows a front view assembled device of FIG. 5.

FIG. 10 shows a perspective view of an arteriovenous banding device, according to one implementation.

FIG. 11 shows a side view of the device of FIG. 10.

FIG. 12 shows a front view of the device of FIG. 10.

FIG. 13 shows a back view of the device of FIG. 10.

FIG. 14 shows a side view of the screw and magnet, according to one implementation.

FIG. 15 shows an image of a prototype screw and magnet assembly, according to one implementation.

FIG. 16, FIG. 17, FIG. 18, and FIG. 19 each show images of a prototype arteriovenous banding device, according to one implementation.

FIG. 20 shows a control system with an example arteriovenous banding device, according to one implementation.

FIG. 21 shows a perspective view of an arteriovenous banding device, according to another implementation.

FIG. 22 shows a perspective view of the device of FIG. 21.

FIG. 23 shows a top view of the device of FIG. 21.

FIG. 24 shows a back view of the device of FIG. 21.

FIG. 25 shows a side view of the device of FIG. 21.

FIG. 26 is an image of a prototype arteriovenous banding device, according to one implementation.

FIG. 27 and FIG. 28 show perspective view of an arteriovenous banding device, according to another implementation.

FIG. 29 shows a side view of the device of FIG. 27.

FIG. 30 shows a perspective view of an arteriovenous banding device, according to another implementation.

FIG. 31 shows a back view of the device of FIG. 30.

FIG. 32 shows a front view of the device of FIG. 30.

FIG. 33 shows a bottom perspective view of the device of FIG. 30.

FIG. 34 shows a perspective view of an arteriovenous banding device, according to another implementation.

FIG. 35 shows a side view of the device of FIG. 34.

FIG. 36 shows a perspective view of the device of FIG. 34.

FIG. 37 and FIG. 38 show perspective views of a system including two of the devices of FIG. 34 interconnected with each other, according to one implementation.

FIG. 39 shows a cross section of the system of FIG. 37.

FIGS. 40 and 41 show perspective views of a system including two of the devices of FIG. 21 interconnected with each other, according to one implementation.

FIG. 42 shows a cross section of the system of FIG. 37.

FIG. 43 shows an image of a disassembled prototype arteriovenous fistula banding device, according to one implementation.

FIG. 44 shows an image of the device of FIG. 43 as assembled.

FIG. 45 shows an image of the device of FIG. 44 in a curved configuration.

FIG. 46 shows an image of the device of FIG. 44 adjacent to an external magnet in a human hand, according to one implementation.

FIG. 47 shows an image of the device of FIG. 44 in a small diameter configuration and surrounding a flexible tube.

FIG. 48 shows an image of the same device of FIG. 47 from a front view.

FIG. 49 shows a perspective view of an external driver device, according to one implementation.

FIG. 50 shows a top view of the device of FIG. 49.

FIG. 51 shows a perspective view of the external driver device of FIG. with half of the housing removed, according to one implementation.

FIG. 52 shows a side view of the device of FIG. 51.

FIG. 53 shows a top view of the device of FIG. 51.

FIG. 54 shows a front view of the device of FIG. 51.

FIG. 55 shows a back view of the device of FIG. 51.

FIG. 56 shows an image of a prototype external driver device, according to one implementation.

FIG. 57 shows an image of a prototype external driver device, according to one implementation.

FIG. 58 shows an image of the disassembled device of FIG. 57.

FIG. 59 shows a front view and FIG. 60 shows a perspective view of an alternative implementation of a handle, according to one implementation.

FIG. 61 shows a front view and FIG. 62 shows a perspective view of an alternative implementation of a handle, according to one implementation.

FIG. 63 depicts a cross section of a system for driving an arteriovenous fistula banding device, according to one implementation.

FIG. 64 depicts a perspective view of the arteriovenous fistula banding device of FIG. 63.

FIG. 65 and FIG. 66 show a system for driving an arteriovenous fistula banding device, according to one implementation.

FIG. 67 shows a perspective view image of a prototype testing device, according to one implementation.

FIG. 68 shows a front view image of the testing device of FIG. 67.

FIG. 69 shows a bottom view image of the testing device of FIG. 67.

FIG. 70 shows a perspective view of a testing device with an arteriovenous banding device and an external driver installed thereon for testing, according to one implementation.

FIG. 71 shows a front view of the devices of FIG. 70.

FIG. 72 shows a bottom perspective view of the devices of FIG. 70.

FIG. 73 shows a cross section of the devices of FIG. 70 across section AA.

FIG. 74 shows a cross section of the devices of FIG. 70 across section BB.

FIG. 75 and FIG. 76 show images of the manufactured prototypes of a testing device with an external driver and an arteriovenous banding device installed thereon for testing, according to one implementation.

FIG. 77 depicts a flowchart which describes a method of controlling blood flow through an arteriovenous fistula, according to one implementation.

### DETAILED DESCRIPTION

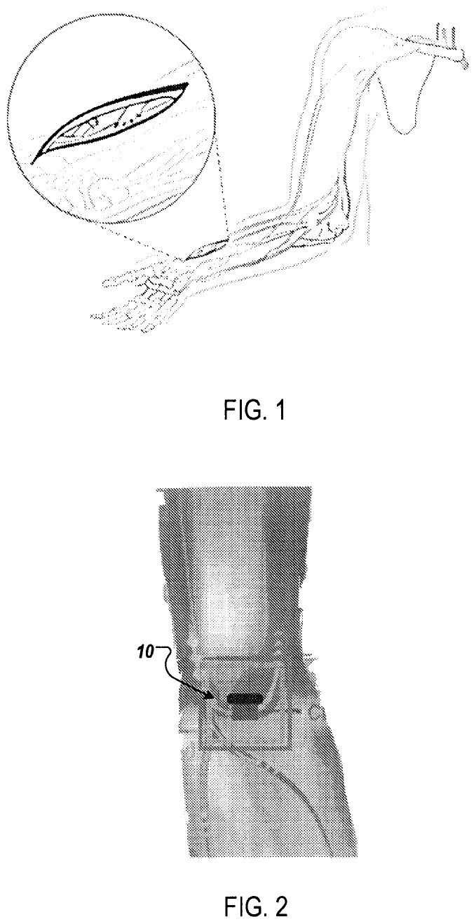

Referring generally to the figures, systems for controlling blood flow through an arteriovenous fistula (AVF) are shown, according to various implementations. Patients with end-stage renal disease undergoing hemodialysis (HD) may require a functional vascular access, which may include arteriovenous fistula (AVF), arteriovenous graft, or central venous catheter. AVFs may be preferred because they have superior longevity, lower infection rate resulting in improved patient survival and lower cost when compared to other types of access. FIG. 1 provides an illustration of a surgical creation of an arteriovenous fistula, according to one implementation.

AVFs are made by surgically connecting a vein to an artery. An AVF may have at least 600 cc/min of blood flow and may be >6 mm wide. When this is done, the flow within the vein immediately increases from ˜20-60 cc/min to ˜200 cc/min since it is a low-resistance circuit. In one scenario the flow with gradually increase over the following few weeks to achieve a goal of >600 cc/min. However, because of this sudden initial increase in blood flow, and the turbulence created by it, the vein's wall usually adapts by trying to become thicker (neo-intimal hyperplasia). This will prevent the blood flow from increasing further in the AVF, which limits its use. The devices, systems, and methods described herein help to solve this problem of limited use of AVFs.

FIG. 2 illustrates an example device 10 implanted in a patient at a desired point on an AVF, according to one implementation. The device 10 may be implanted during the AVF grafting procedure or implanted around an existing graft at a later time. The device 10 includes a band wrapped around the AVF and configured to act like a faucet that is loosened during hemodialysis and tightened outside hemodialysis. This allows for high blood flow through the AVF only when needed/desired. Therefore, complications due to the high flow rates are limited, and a closer-to-normal cardiac output is maintained when the patient is outside of dialysis sessions. Additionally, the systems, methods, and devices herein described may help to increase AVF maturation rates by facilitating the creation of larger arteriotomy area. The example device 10 of FIG. 2 can act as a safety valve to reduce high flow when needed.

The device 10 of FIG. 2 may include numerous variations in size, number, function, placements, controls, and implementation. As such, the following figures and corresponding descriptions provide examples of devices for controlling blood flow through an AVF and related systems.

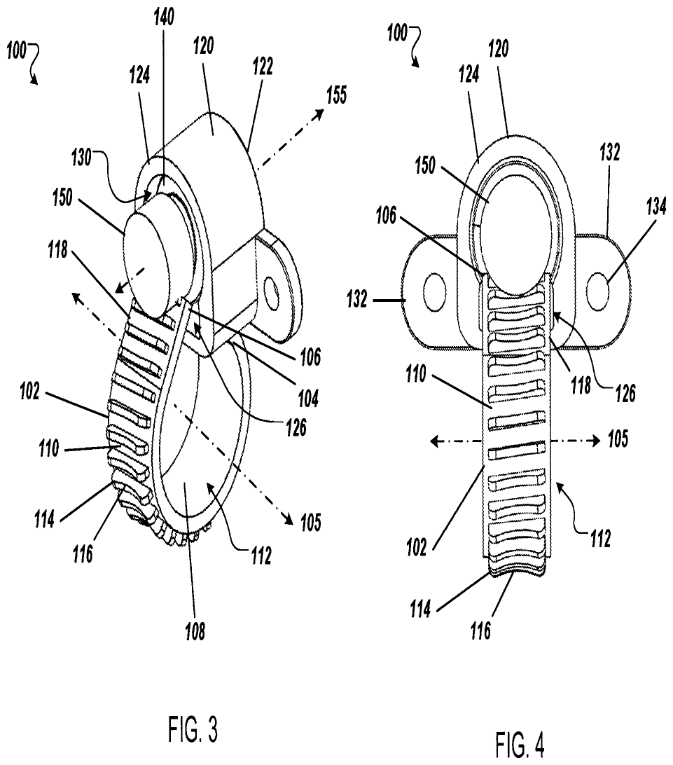

FIG. 3 shows a perspective view of an arteriovenous fistula banding device 100, according to one implementation. FIG. 4 shows a front view of the device 100. The device 100 includes a band 102, a housing 120, a screw 140, and an actuator 150. The band 102 includes a first end 104 and a second end 106 spaced apart from the first end 104. The band 102 includes an inner band surface 108 and an outer band surface 110 opposite from the inner band surface 108. The band 102 extends circumferentially around a central axis 105 to define to define a central channel 112 having a first diameter. Specifically, the inner band surface 108 defines the central channel 112. While most of the band 102 has a curved shape, the second end 106 of the band 102 includes a linear portion 118.

The first diameter of the central channel 112 is 10 mm. However, in other implementations, the first diameter may be in the range from 3 mm to 10 mm (e.g., 3 mm, 5 mm, 7 mm, 10 mm). As will be described further, the first diameter of the central channel 112 is adjustable from 3 mm to 10 mm. The band 102 is sized to allow for the changes in diameter. For example, a change from 3 mm to 10 mm requires a circumference change of roughly 22 mm, meaning that the band 102 must have at least 22 mm of “slack” beyond that surrounding the smaller diameter configuration.

The band 102 further includes a plurality of teeth 114 protruding from the outer band surface 110. The plurality of teeth 114 are disposed at a slight angle with respect to the direction of the width of the band 102, as seen in FIG. 4 (e.g., to engage with a worm gear). Each of the plurality of teeth 114 also include a central cutout 116 to accommodate the actuator 150. The width of the band 102 as measured in a direction parallel to the central axis 105 is 5 mm. However, in other implementations, the width of the band may be in the range from 5 mm to 15 mm (e.g., 5 mm, 7 mm, 10 mm, 12 mm, or 15 mm). Changes in the width of the band 102 can affect the pressure put on the arteriovenous fistula when compressed, including distributing the pressure over a larger area. Furthermore, the thickness of the band 102, measured from the inner band surface 108 to the outer band surface 110, may vary from 0.4 mm to 1.4 mm. The thickness of the band 102 can affect the flexibility of the band and the ease of contracting the band 102 to a smaller diameter. In other implementations, the thickness of the band may be larger or smaller and may be affected by the type of material(s) used in the band. In some implementations, the thickness of the band ranges from 0.2 mm (e.g., for a thin strong fabric) up to 4 mm (e.g., for silicon or another flexible material).

The plurality of teeth 114 of the band 102 extend only from a distal portion of the band 102 (e.g., from the second end 106 to a point in between the first end 104 and the second end 106) such that a proximal portion of the band 102 (e.g., from the first end 104 to a point in between the first end 104 and the second end 106) includes no teeth 114. Because the proximal portion of the band 102 includes no teeth, the screw 140 is unable to engage with or pull that portion of the band 102. Thus, the proximal portion of the band 102 defines a minimum diameter of the central channel 112.

In some implementations, the band 102 further includes a mechanical stop on the second end 106 of the band 102 to define in maximum diameter of the central channel 112. In either case, including a mechanical means of limiting the diameter of the central channel 112 prevents disconnection of the band 102 from the housing 120 or squeezing the arteriovenous fistula too small. In some implementations, a physical switch or secondary magnet mechanism prevents free rotation of the magnet and thus maintains the band in a particular configuration until activated by an external magnet. These and other safety mechanisms may be implemented in a variety of implementations of the device 100.

The housing 120 includes a first side 122 and a second side 124 opposite and spaced apart from the first side 122. The first side 122 is coupled to the first end 104 of the band 102. The second side 124 of the housing 120 defines a main opening 126 which accepts the second end 106 of the band 102 after it wraps around the central channel 112. Thus, the housing 120 is disposed outside of and adjacent to the central channel 112. In some implementations, the housing 120 has a thickness of 2-5 mm.

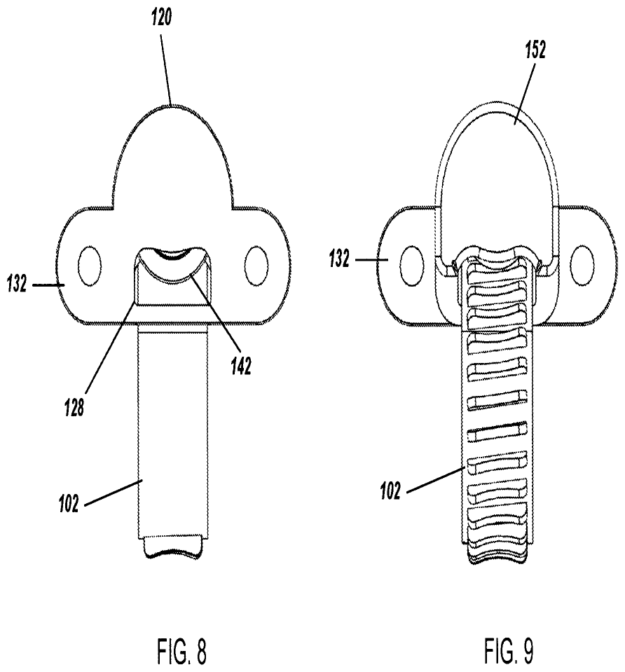

The housing 120 further defines a main cavity 130 which extends from the main opening 126 on the second side 124 to the first side 122 of the housing. The first side 122 of the housing further defines a housing slot 128 (more clearly visible in FIG. 8) configured to accept the second end 106 of the band 102 after it wraps around the central channel 112 and passes through the main opening 126 and the main cavity 130.

The housing 120 further includes a pair of suture protrusions 132 extending out from the first side 122 of the housing 120 in a direction transverse to the band 102. The suture protrusions 132 are disposed outside of the central channel 112. Each of the suture protrusions 132 defines a suture hole 134 configured to accept a surgical suture (e.g., for implanting the device 100).

The screw 140 (e.g., a worm gear) is disposed within the main cavity 130 of the housing 120 such that the main opening 126 accepts the screw 140 into the main cavity 130. The screw 140 includes at least one thread 142 wrapped around the screw 140. The at least one thread 142 is disposed adjacent to or abutting the band 102 when the second end 106 of the band 102 enters the main cavity 130 though the main opening 126. Specifically, the plurality of teeth 114 of the band 102 are engageable with the at least one thread 142 of the screw 140. The screw 140 includes a first end 144 adjacent to the first side 122 of the housing and a second end 146 adjacent to the second side 124 of the housing 120.

The actuator 150 is coupled to the screw 140 (e.g., via adhesive resin or integrally formed together). Specifically, the actuator 150 is coupled to and/or disposed on the second end 146 of the screw 140. The actuator 150 in device 100 is disposed entirely outside of the main cavity 130 of the band 102. However, in other implementations, the actuator 150 may be partially disposed within the main opening 126.

The actuator 150 of device 100 is a magnet 150 (e.g., a diametrically-magnetized magnet) rotatably disposed adjacent to the main opening 126 of the housing 120. The magnet 150 and screw 140 are configured to rotate about a screw axis 155. However, in other implementations, the actuator is a motor with a shaft coupled to the screw. In some implementations, the motor actuator may further include an encoder, internal battery, and a transmission means (e.g., a Bluetooth antenna) to communicate with a remote controller. The motor may be, for example, a Micromo 3 mm diameter motor with a length of 8 mm.

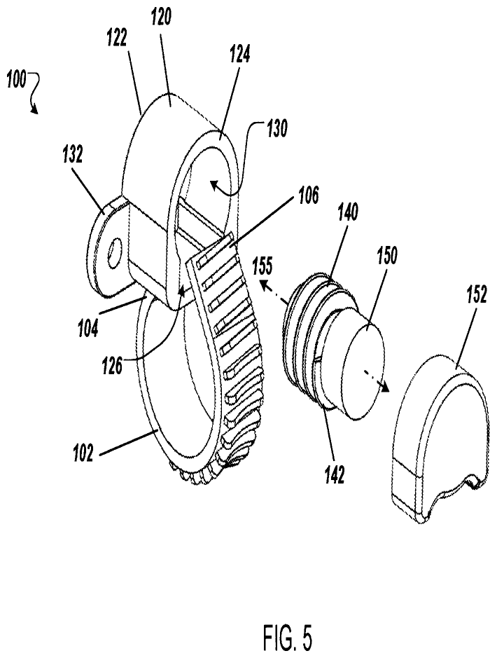

FIG. 5 shows and exploded view of the device 100 of FIGS. 3 and 4. In FIG. 5, the screw 140 is shown outside of the housing 120 such that the main cavity 130 is empty. The device 100 of FIG. 5 also includes a magnet cover piece 152 configured to cover and house the magnet 150. The magnet cover piece 152 is separably couplable to the housing 120 at the first side 122 (e.g., via adhesive resin or mechanical coupling).

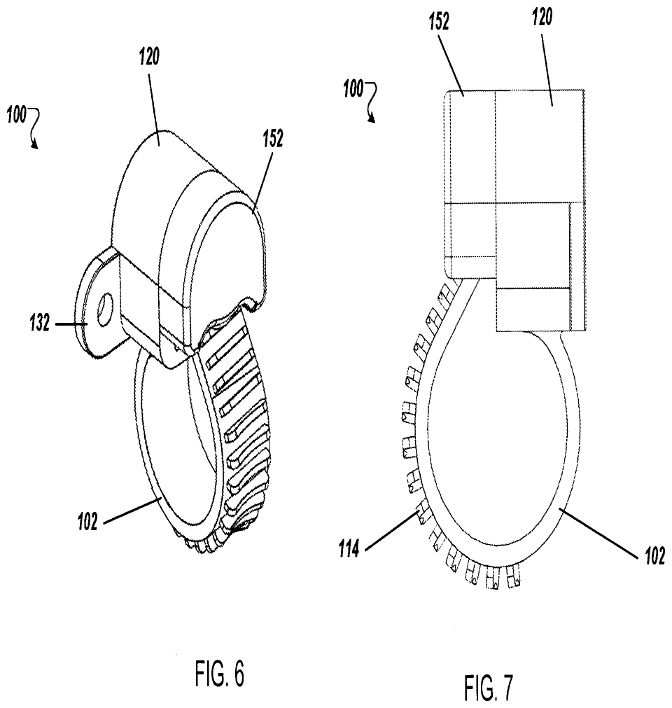

FIG. 6 shows a perspective view of the device 100 of FIG. 5, and FIG. 7 shows a side view of the device 100 of FIG. 5, each showing the placement of the magnet cover piece 152. FIG. 8 shows a back view of the device 100 of FIG. 5, wherein the housing slot 128 is visible on the first side 122 of the housing 120. FIG. 9 shows a front view of the device 100 of FIG. 5.

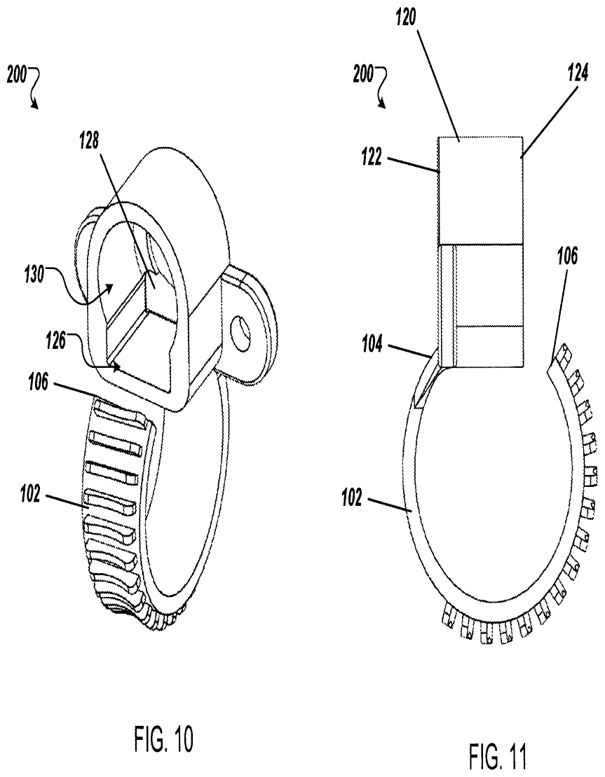

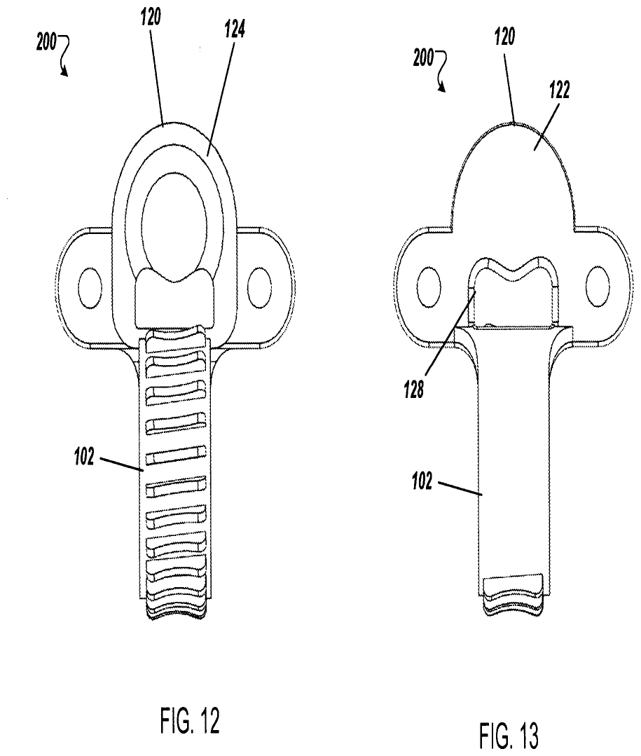

FIG. 10 shows a perspective view of a device 200. Additionally, FIG. 11 shows a side view, FIG. 12 shows a front view, and FIG. 13 shows a back view of the device 200 of FIG. 10. The device 200 is substantially similar to device 100 of FIGS. 3-9. However, instead of including a linear portion 118 on the second end 106 of the band 102 (as in device 100), the band 202 of device 200 is curved along the entire length of the band 202 from the first end 204 to the second end 206.

Each device 100 and device 200 may be 3D printed in a curved configuration such that the band 102 or band 202 is biased towards the curved configuration. The devices described herein may also be printed in a flat configuration and then manipulated into a curved configuration as shown. In other implementations, the devices described herein are manufactured in a mold (e.g., a two-sided resin mold). The device 100 and device 200 may comprise a flexible resin (e.g., Flexible 80A resin), silicone, PTFE, or any other biocompatible material. In some implementations, the band comprises Flexible 80A resin.

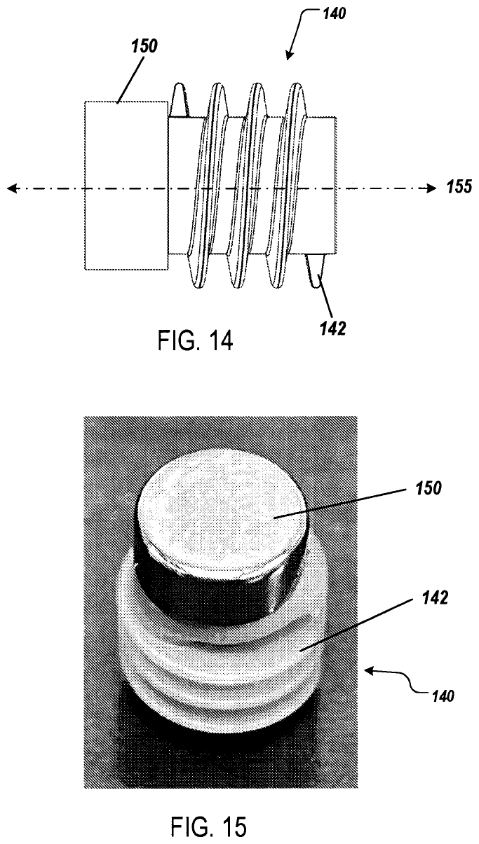

FIG. 14 shows a side view of the screw 140 and magnet 150. The at least one thread 142 of the screw 140 is shown. In example implementations, the screw 140 may have an inner core diameter of 0.2 inches and an outer thread diameter of 0.38 inches. In example implementations, the screw 140 may have a length of 0.25 inches. In other implementations, the screw may have an inner diameter as small as 0.1 inches and an outer thread diameter of 0.2 inches. In some implementations, longer threads can be used to ensure good engagement with the band. Similarly, in example implementations, the magnet 150 may have a diameter of ⅛ inch or ¼ inch and a heigh of ⅛ inch. In other implementations, the magnet diameter could be more than ¼ inch (e.g., ⅜ inch). In some implementations, the magnet height can be ¼ inch depending on the coupling force needed for that implementation.

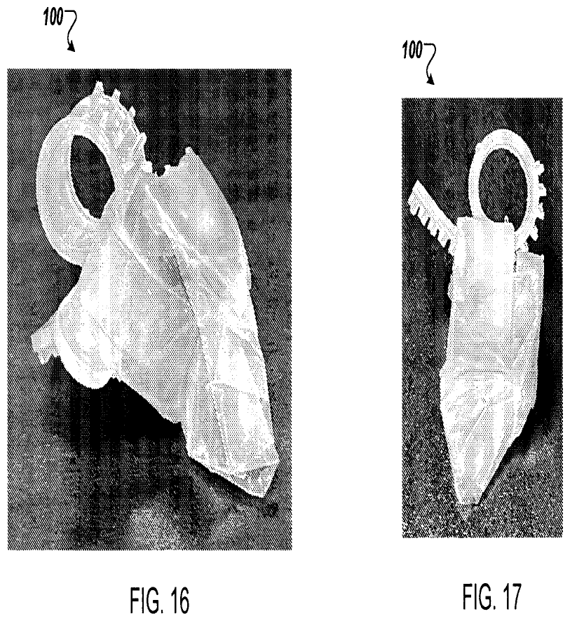

FIG. 15 shows an image of a prototype screw and magnet assembly, according to one implementation. The magnet 150 of FIG. 16 has a diameter of ¼ inch and is coupled to the screw 140 via adhesive resin.

FIGS. 16, 17, 18, and 19 show images of a prototype of the device 100. Specifically, FIG. 16 shows a perspective view image of the prototype device 100, FIG. 17 shows a side view image of the prototype device 100, FIG. 18 shows a perspective view of the prototype device 100, and FIG. 19 shows a side view image of the prototype device 100. The difference between FIGS. 16 and 17 compared to FIGS. 18 and 19 is that FIGS. 16 and 17 show a contracted or small diameter configuration of the device 100 while FIGS. 18 and 19 show an expanded or large diameter configuration of the device 100. Movement between the contracted and expanded configurations is described with reference to device 100 of FIGS. 3-9 and the corresponding reference numbers.

In use, the device 100 is implanted within a patient (e.g., implanted in the arm of a patient adjacent to an arteriovenous fistula site). The device 100 may be secured in place by sutures engaging with the device 100 via the suture holes 134 of the suture protrusions 132. The screw 140 and magnet 150 are inserted through the main opening 126 of the housing 120. The screw 140 is rotatably disposed in the main cavity 130 while the magnet 150 is disposed outside of the housing 120 and attached to the screw 140.

The second end 106 of the band 102 is wrapped around a portion of the arteriovenous fistula until it reaches the second side 124 of the housing 120, thus defining the central channel 112 with the arteriovenous fistula disposed therein. The second end 106 of the band 102 is inserted through the main opening 126 and into the main cavity 130 adjacent to the screw 140. The plurality of teeth 114 on the outer band surface 110 of the band 102 abut and engage with the at least one thread 142 of the screw 140. The screw 140 with the magnet 150 may be rotated slightly to accept and secure the second end 106 of the band 102 within the main cavity 130. Then, the magnet cover piece 152 is secured to the housing 120 over the magnet 150.

When actuated, magnet 150 rotates about the screw axis 155 and causes rotation of the screw 140 about the screw axis 155 as well. Rotation of the screw 140 causes the at least one thread 142 to engage with the plurality of teeth 114 of the band 102. As the screw 140 and the at least one thread 142 rotates in a first direction (e.g., clockwise), the second end 106 of the band 102 moves further into the main cavity 130 of the housing 120 from the second side 124 to the first side 122. The second end 106 of the band 102 can exit the main cavity 130 through the housing slot 128 defined by the first side 122 of the housing 120. As the screw 140 rotates in the first direction and moves the band 102 through the housing 120, the diameter of the central channel 112 reduces. Thus, once the second end 106 of the band 102 exits the housing 120 via the housing slot 128, the second end 106 of the band moves away from the first end 104 of the band 102, thus reducing the diameter of the central channel 112. For the description of device 100, the first diameter will be the contracted configuration with the smaller diameter, shown in FIGS. 16 and 17. However, the “first diameter” of the central channel 112 could be the smaller diameter of the contracted configuration or the larger diameter or the expanded configuration.

When actuated to move in a second direction (e.g., counter-clockwise), the magnet 150 causes the screw 140 to rotate to expand the diameter of the central channel 112. The second end 106 of the band 102 moves towards the first end 104 of the band 102 and back through the housing slot 128 and into the main cavity 130 until a desired second diameter is reached.

The device 100 may be implanted in a patient and placed in the contracted configuration (first diameter of the central channel 112) as shown in FIGS. 16 and 17. In this configuration, the arteriovenous fistula (AVF) is compressed to have an inner diameter smaller than in its neutral state, which reduces the blood flow through the arteriovenous fistula. The smaller inner diameter (e.g., 3 mm) is preferable for everyday operation so that complications from high blood flow through the AVF are avoided.

Once a patient is ready to undergo hemodialysis, the device 100 is moved from the contracted configuration to the expanded configuration of FIGS. 18 and 19. The larger second diameter (e.g., 10 mm) of the central channel 112 provides for a higher blood flow through the AVF which is preferable for a dialysis treatment. During dialysis, the device 100 may be adjusted. That is, the diameter of the central channel 112 may be adjusted (e.g., discretely or continuously) to adjust the blood flow through the AVF. The patient may be monitored to ensure the dialysis is functioning as expected, and the blood flow rate may be reduced or increased as needed. Once complete, the device 100 is returned to the first diameter or the contracted configuration.

Example Device and Control System

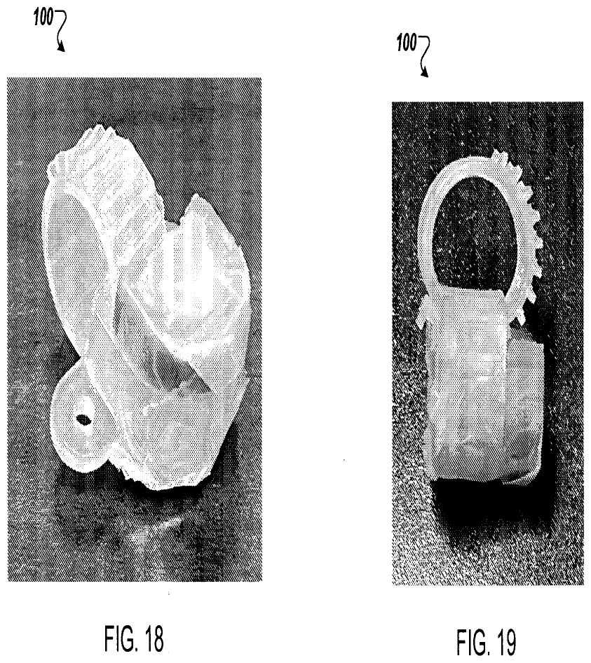

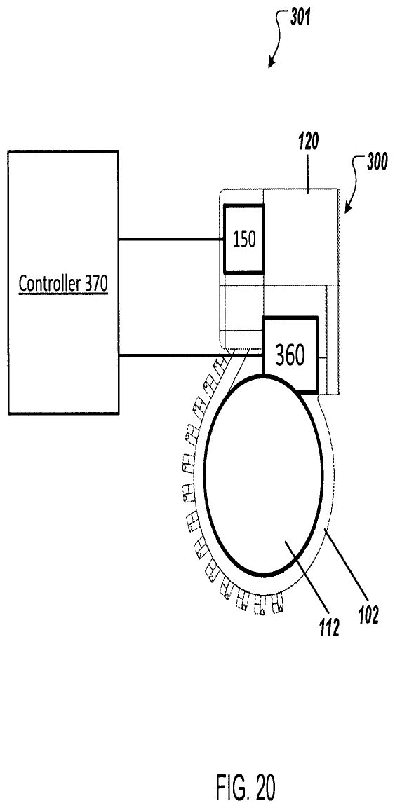

FIG. 20 shows an example control system 301 with an example device 300 which is substantially similar to device 100 of FIGS. 3-9. However, device 300 further includes a flow sensor 360 disposed adjacent to the central channel 112. The flow sensor 360 is configured to measure fluid flow through the central channel 112. The flow sensor 360 may be disposed partially within the housing 120 and may include or be connected to a transceiver (e.g., a Bluetooth antenna) to transmit fluid flow data. The flow sensor 360 may be a portion of the device 100 or an attachment as part of the control system 301.

Control system 301 further includes a controller 370 configured to receive measured fluid flow data from the flow sensor 360. For example, the flow sensor 360 may transmit measured fluid flow data to the controller 370 remotely or by wired connection. The controller 370 may be disposed within the housing 120 of the device 300, or the controller 370 may be disposed within an external device nearby the patient and the device 300. The location or implementation of the controller 370 may depend on the procedure to be performed.

The controller 370 is configured to compare the measured fluid flow to a predetermined fluid flow (e.g., a desired blood flow stored in a memory on the controller). The controller 370 uses the comparison to drive the actuator or magnet 150, which turns the screw (e.g., screw 140) to adjust the band 102 and thus the diameter of the central channel 112. This process may be continuous (e.g., for the duration of a dialysis treatment) or discrete (e.g., set points throughout a dialysis treatment). The feedback loop formed between the device 300, the flow sensor 360, the controller 370, and the actuator 150 allows for accurate and fast control of blood flow through the arteriovenous fistula.

Alternative Embodiments

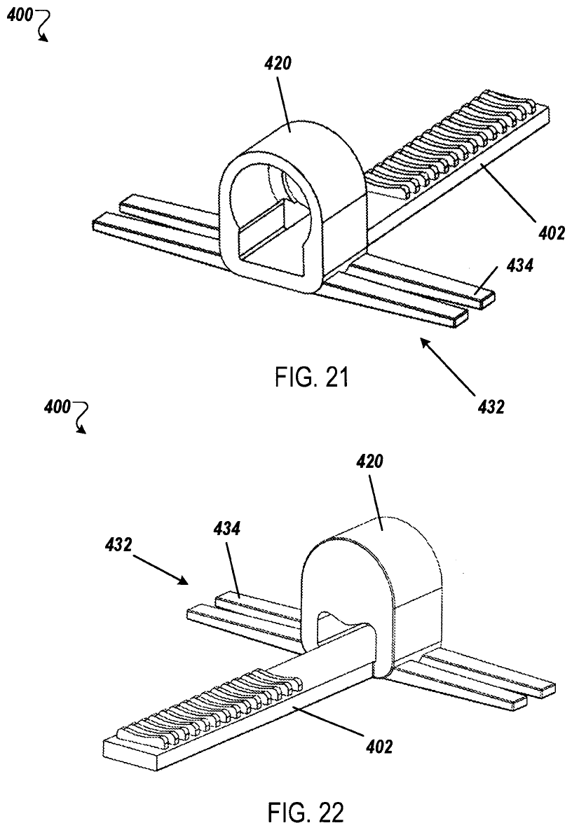

FIGS. 21-26 show a device 400. The device 400 is similar to device 100 of FIGS. 3-9, however, device 400 is manufactured with the band 402 in a flat configuration. Device 400 also includes a different implementation of the suture protrusions. The suture protrusions 432 of device 400 include two pairs of protruding wings 434 extending out from the housing 420 at an angle transverse to the direction of extension of the band 402. The wings 434 of the suture protrusions 432 provide attachment points for suturing the device 400 in a desired location in a patient adjacent to an arteriovenous fistula.

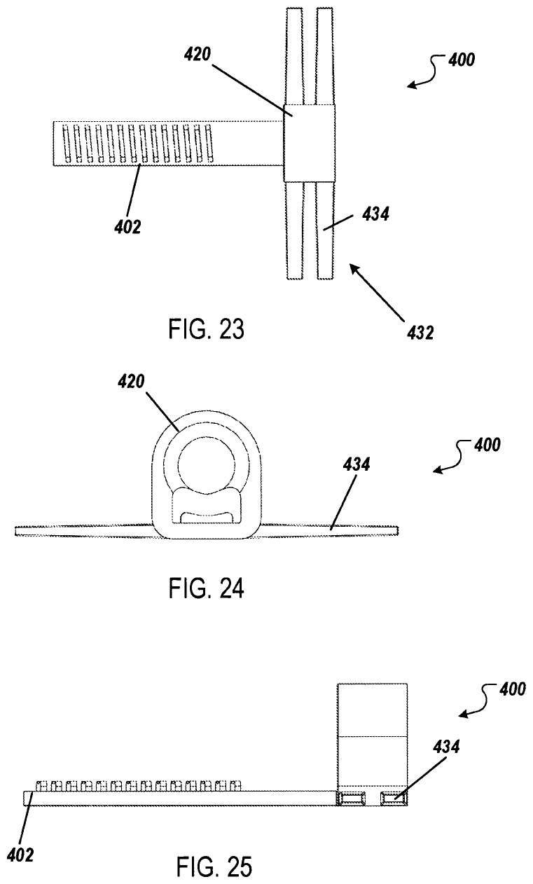

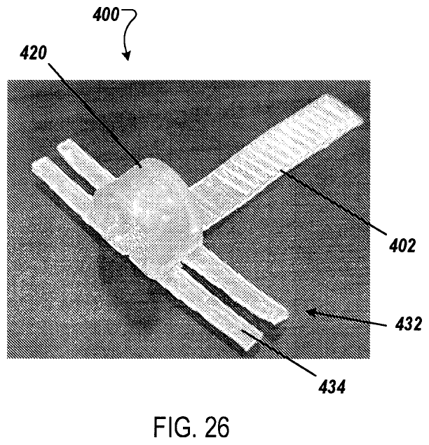

FIG. 21 and FIG. 22 show perspective views of the device 400. FIG. 23 provides a top view of the device 400, FIG. 24 provides a back view of the device 400, and FIG. 25 provides a side view of the device 400. FIG. 26 is an image of a prototype of device 400.

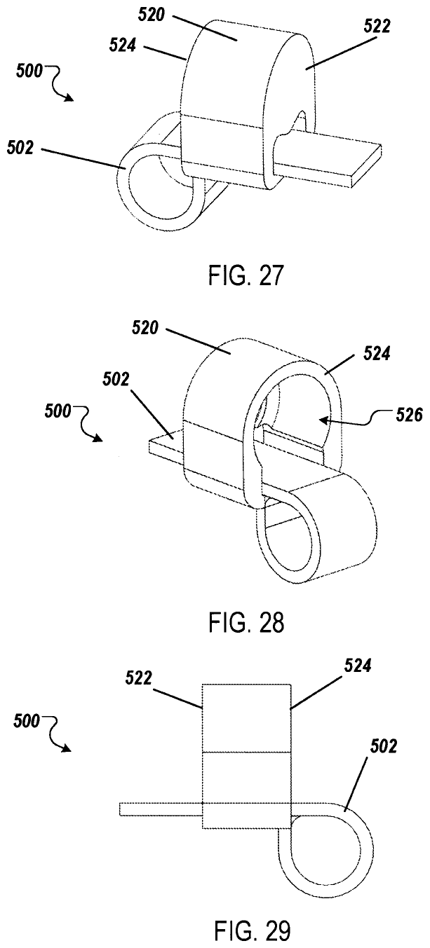

FIGS. 27 and 28 show perspective views of a device 500, and FIG. 29 shows a side view of the device 500. The device 500 is similar to device 100 of FIGS. 3-9, however, device 500 includes a band 502 is coupled to the second side 524 of the housing (rather than the first side 522). The band 502 extends out from the housing 520 at an angle perpendicular to the second side 524 of the housing (e.g., downward from the housing 520). The band 502 wraps back around to the second side 524 to enter the main opening 526 of the housing. Thus, device 500 includes a tighter curvature of the band 502 compared to device 100.

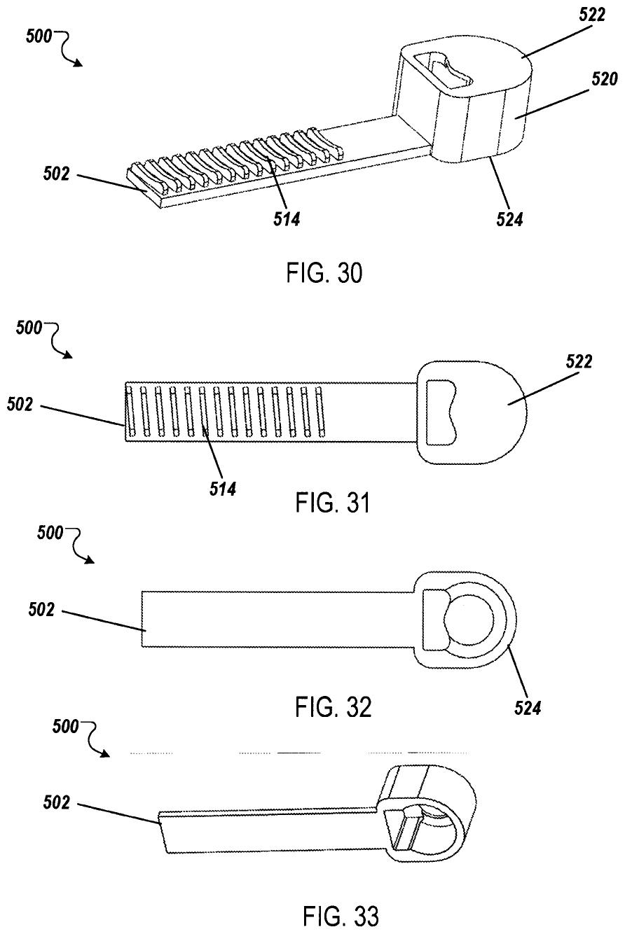

FIGS. 30-33 show a variation on device 500 in the flat configuration rather than the curved configuration. Additionally, device 500 of FIGS. 30-33 includes a plurality of teeth 514 on the band 502. The device 500 in FIGS. 30-33 can curve around in the same manner as device 500 in FIGS. 27-29 to create a tight radius curve of the band 502.

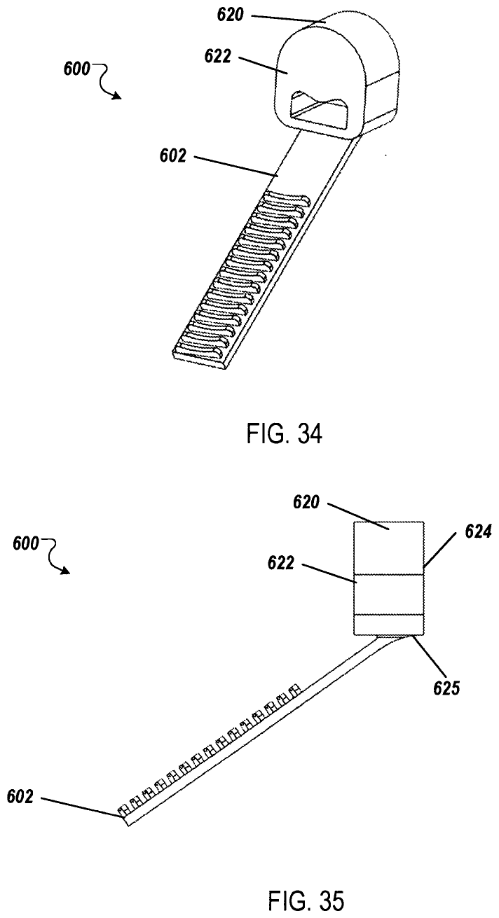

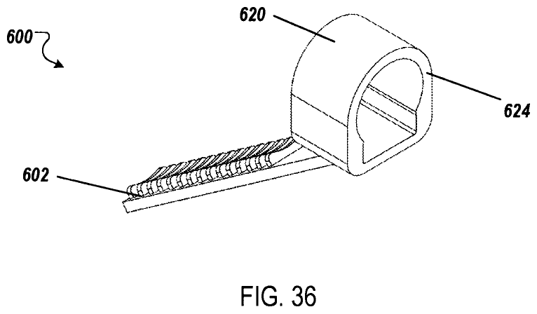

FIG. 34-36 show a device 600. FIG. 34 provides a perspective view, FIG. 35 provides a side view, and FIG. 36 shows a different perspective view of the device 600. The device 600 is substantially similar in structure and function to the previously described devices (e.g., device 100 or device 500). However, the band 602 of device 600 extends from a bottom side 625 of the housing 620 at an angle with respect to the bottom side 625, the first side 622, and the second side 624 of the housing 620.

Example System Functionality

The following systems, devices, and examples describe and depict interconnected devices which depict the geometry and placement of various components. Two devices are often shown where the band of one device enters the housing of another to interact with the screw. While a two-device implementation is possible, these models primarily are provided to show the specific interaction and the interface between the plurality of teeth of the band with the screw.

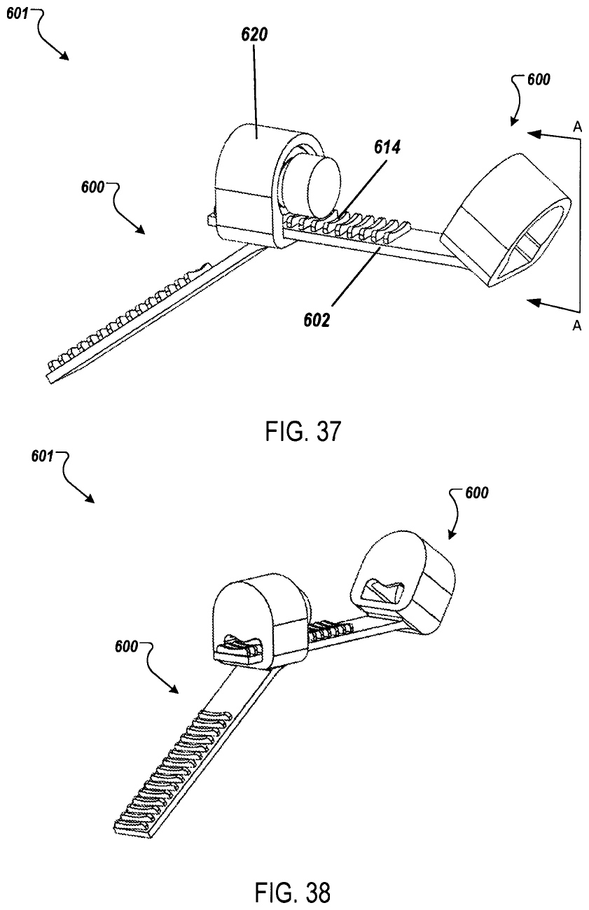

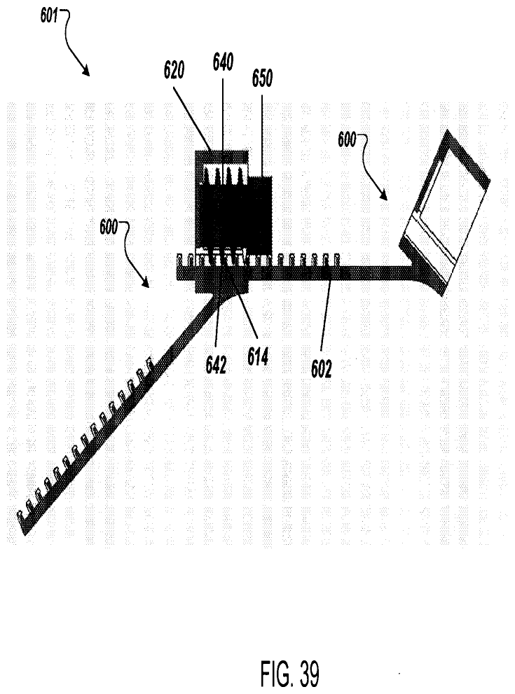

FIGS. 37 and 38 show perspective views of a system 601 including two of the devices 600 interconnected with each other along with a screw 640 and magnet 650. FIG. 39 provides a cross-section view of the system 601 along line A-A in FIG. 37. FIG. 39 shows how the screw 640 with the at least one thread 642 abuts the plurality of teeth 614 of the band 602 within the housing 620.

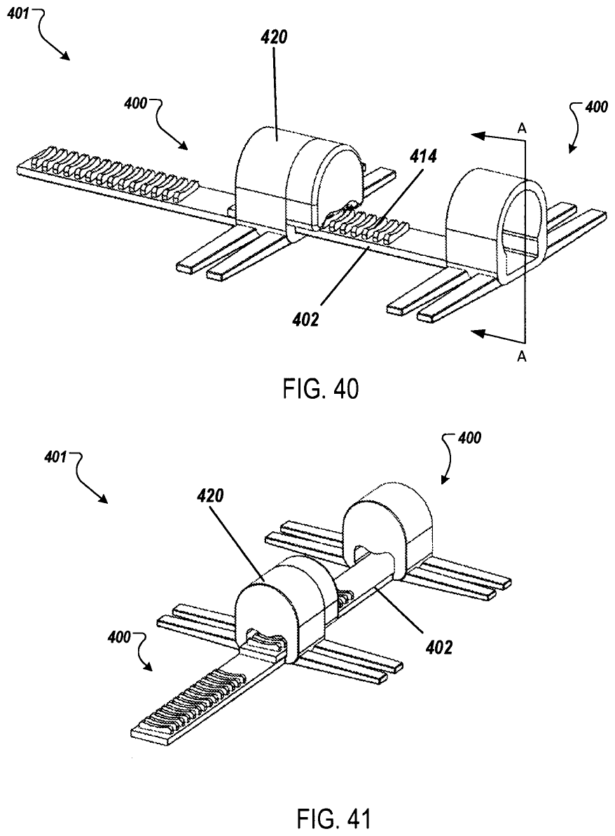

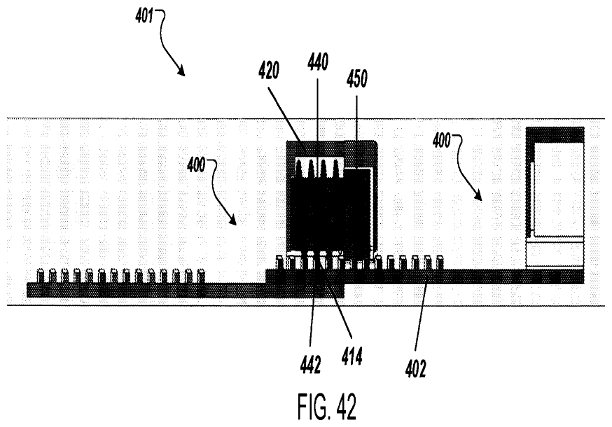

FIGS. 40 and 41 show perspective views of a system 401 including two of the devices 400 interconnected with each other along with a screw 440 and magnet 450. FIG. 42 provides a cross-section view of the system 401 along line A-A in FIG. 40. FIG. 42 shows how the screw 440 with the at least one thread 442 abuts the plurality of teeth 414 of the band 402 within the housing 420.

Example Benchtop Prototype

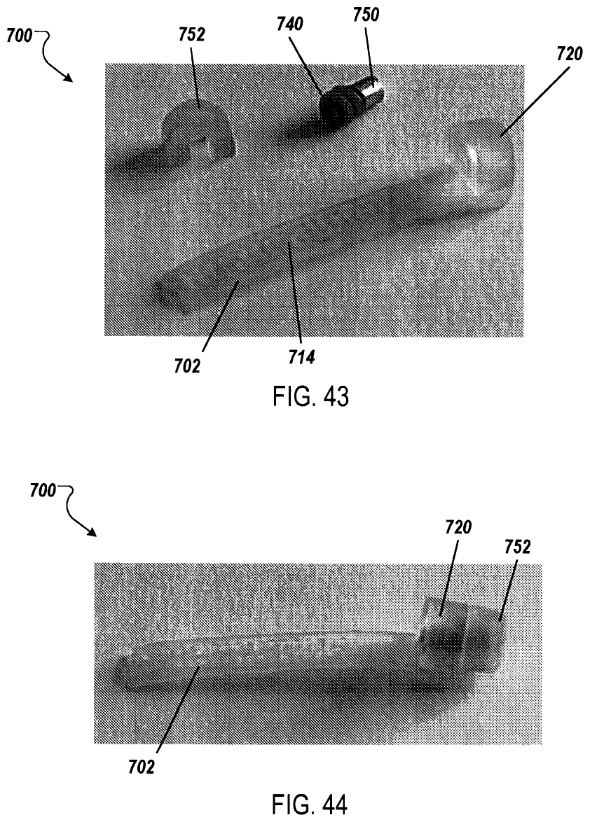

FIGS. 43-48 provide images of a benchtop prototype device 700, according to another implementation. The device 700 is substantially similar in structure and function as device 100; however, device 700 does not include suture protrusions extending from the housing 720. The device 700 does include a band 702 with a plurality of teeth 714 extending from the housing 720. A screw 740 coupled to a magnet 750 is disposed within the housing 720 and the magnet 750 is covered with the magnet cover piece 752. FIG. 43 shows a disassembled version of the device 700 while FIG. 44 shows an assembled version.

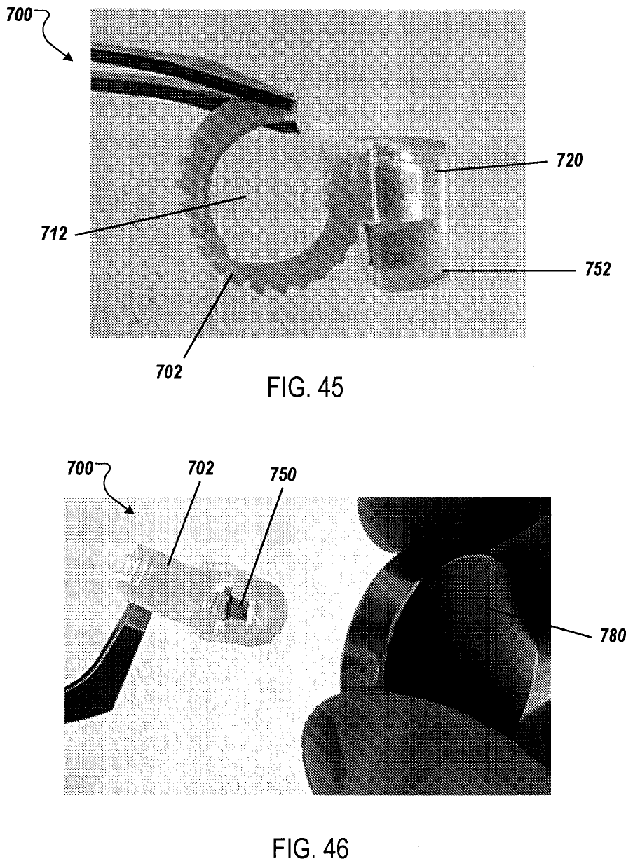

FIG. 45 shows the device 700 with the second end 706 of the band 702 engaged with the screw 740 and disposed within the housing 720. The band 702 thus defines the central channel 712. The device 700 of FIG. 45 is being suspended by tweezers.

FIG. 46 shows the device 700 adjacent to an external magnet 780 (e.g., a diametrically-magnetized magnet). The external magnet 780 is larger in diameter than the magnet 750 of the device 700. For example, the magnet 750 may have a diameter of between ⅛ inch and ⅜ inch while the external magnet 780 may have a diameter of 1 inch with a thickness of ¼ inch or ½ inch thickness. In other implementations, the external magnet may be larger or smaller depending on the coupling force needed for that implementation.

An experiment was performed where the external magnet 780 was rotated about its axis nearby the device 700 with the axis of the magnet 750 (e.g., screw axis 155) substantially aligned with the axis of the external magnet 780. The experiment provided for human manipulation and rotation of the external magnet 780 as a proof of concept. Each of the magnet 750 and the external magnet 780 are diametrically-magnetized magnets (i.e., the poles of the magnet oppose each other along the width of the magnet, rather than along the axis of the magnet). Because they are diametrically-magnetized, rotation of the external magnet 780 can drive rotation of the magnet 750 when they are axially aligned. The results of the experiment showed that rotation of the external magnet 780 did cause a rotation of the magnet 750 which, in turn, caused a rotation of the screw 740. Therefore, the band 702 was pulled into the housing 720 and the diameter of the central channel 712 was reduced.

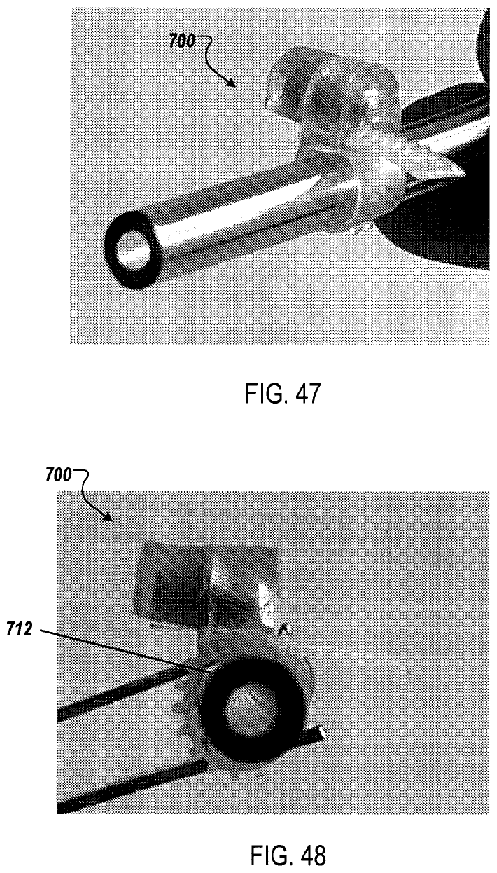

FIG. 47 shows a perspective view image, and FIG. 48 shows a front view image, of the resulting smaller-diameter configuration of the band 702. As shown, a flexible tube is disposed within the central channel 712 to represent the arteriovenous fistula. This benchtop prototype and associated testing proved the viability of the external magnet 780 and its function in driving the magnet 750.

Variations on the distance between the magnets, the size of the magnets, and the materials used in the device 700 can yield various values of torque output. In some implementations, the magnets are sized such that a desired input torque drives rotation of the magnet 750 and screw 740 at a desired output torque.

External Driving System #1

Once an arteriovenous fistula banding device (e.g., device 100) is implanted in a patient, a need arises to reliably and controllable drive the actuator 150 (e.g., magnet 150). While some implementations of the device 100 provide for an internal driving mechanism (e.g., a small motor communicating with a remote control system), provided herein is a system for driving the device 100 having a magnet 150 as the actuator. Specifically, described herein is a system for driving an arteriovenous fistula device (e.g., device 100) by the use of an external driver 800 with a driver magnet 802.

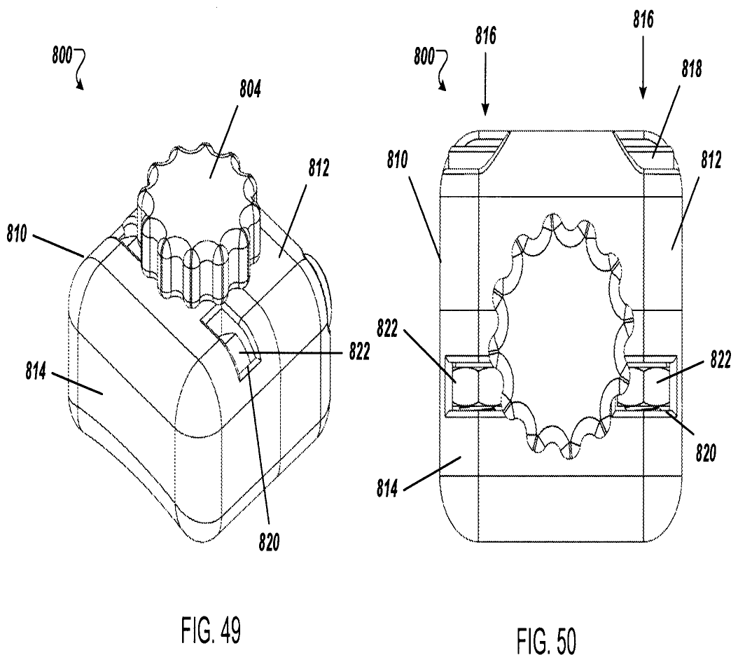

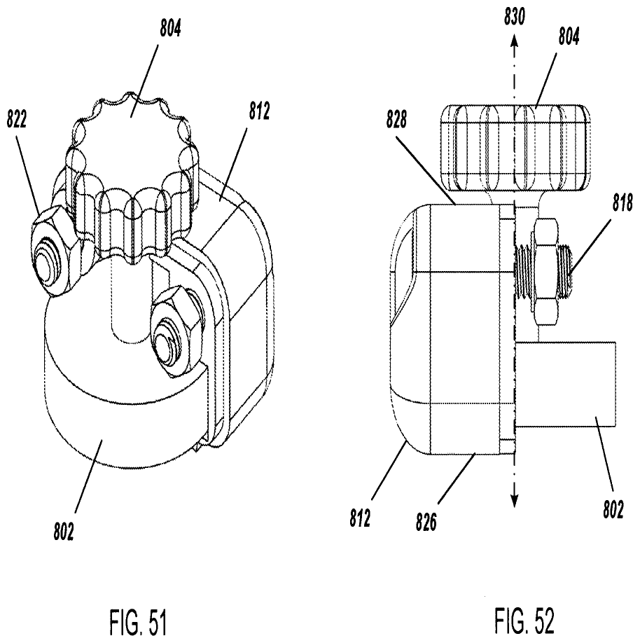

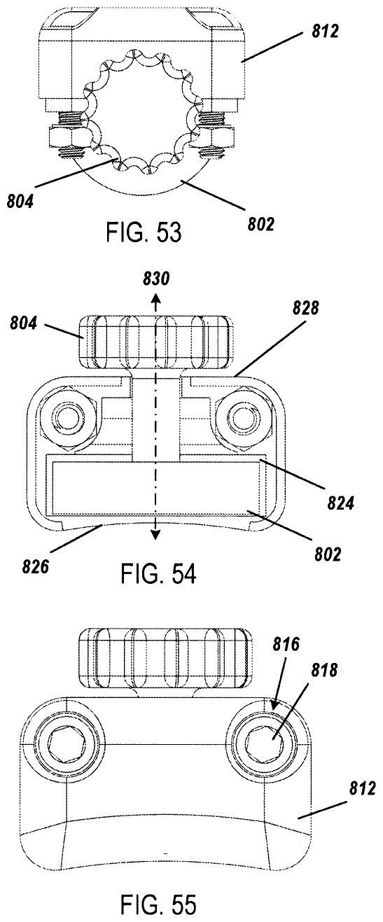

FIG. 49 shows a perspective view, and FIG. 50 shows a top view, of an external driver 800, according to one implementation. The external driver 800 includes an outer housing 810 comprised of halves—a first body 812 and a second body 814. FIGS. 51-55 show the external driver 800 with the second body 814 removed so that internal components are visible. FIG. 51 shows a perspective view of the external driver 800 without the second body 814, while FIG. 52 provides a side view, FIG. 53 provides a top view, FIG. 54 provides a front view, and FIG. 55 provides a back view of the same.

The first body 812 includes two screw slots 816 extending through the first body 812 towards the second body 814. The screw slots 816 are configured to accept a screw 818 therethrough. The second body 814 includes two fastener slots 820 each configured to accept a nut 822. Thus, the first body 812 is coupled to the second body 814 by placing the nuts 822 in the two fastener slots 820 of the second body 814 and placing the screws 818 in the screw slots 816 of the first body 812. The screws 818 engage with the nuts 822 to secure the housing 810 in place. In some implementations, the screws 818, nuts 822, housing 810, and other components of the external driver 800 are made from plastic or other non-ferrous material so that interference with the driver magnet 802 is avoided.

The external driver 800 includes a driver magnet 802 (e.g., the external magnet 780 from the benchtop prototype). The driver magnet 802 is disposed within a magnet slot 824 of the housing 810. The driver magnet 802 is disposed close to a bottom surface 826 of the housing 810. The external driver 800 further includes a handle 804 coupled to the driver magnet 802 (e.g., via adhesive resin). The handle 804 extends through a top surface 828 of the housing 810 to engage with and/or couple to the driver magnet 802. The driver magnet 802 and the handle 804 include a turning axis or a main axis 830 about which they rotate.

In use, an arteriovenous fistula banding device (e.g., device 100) is implanted within a patient wherein the band 102 of the device 100 wraps around an arteriovenous fistula and defines a central channel 112 within which the arteriovenous fistula is disposed. In order to adjust the diameter of the central channel 112, the magnet 150 can be rotated by the external driver 800. Specifically, rotation of the the magnetic field of the driver magnet 802 external to the patient can drive rotation of the magnet 150 implanted within the patient about the screw axis 155.

The external driver 800 is placed on the patient adjacent to the device 100 wherein the bottom surface 826 of the external driver 800 is adjacent to the patient's skin. The device 100 is easily located because of the attraction between the driver magnet 802 and the magnet 150 of the device 100. Once in place, the handle 804 is rotated (e.g., by a medical professional or by a medical device with a controller attached) which, in turn, rotates the driver magnet 802 about the main axis 830. The rotating magnetic field of the driver magnet 802 causes the magnet 150 to rotate which causes the diameter of the central channel 112 to increase or decrease, depending on the direction of rotation. The direction of the rotation, and resultant size change in the diameter of the central channel 112, may be indicated on the external driver 800 or by some external control system.

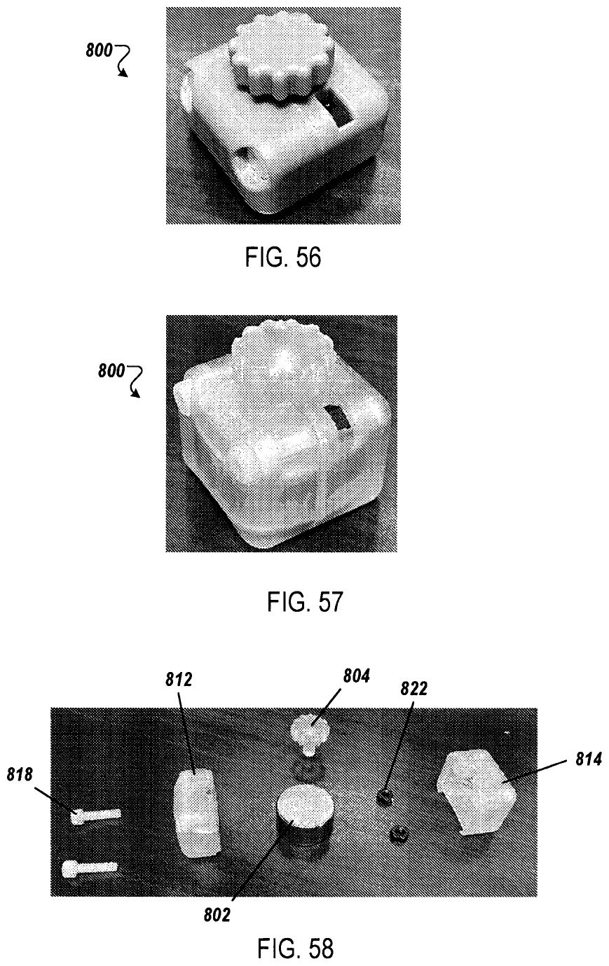

FIGS. 56-58 show images of prototype external drivers 800. FIG. 56 includes an external driver 800 with the housing made of Formlabs photopolymer resin (grey resin). The external driver 800 of FIG. 56 is sized to test a ½ inch thick and 1 inch diameter driver magnet 802. FIG. 57 includes an external driver 800 with the housing made of clear resin. The external driver 800 of FIG. 57 is sized to test a 1 inch thick and 1 inch diameter driver magnet 802. FIG. 58 shows the prototype external driver 800 of FIG. 57 with the components disassembled. In FIGS. 56-58, the housings were made from grey or clear resins while the band and magnet housing were made from Flexible 80A resin.

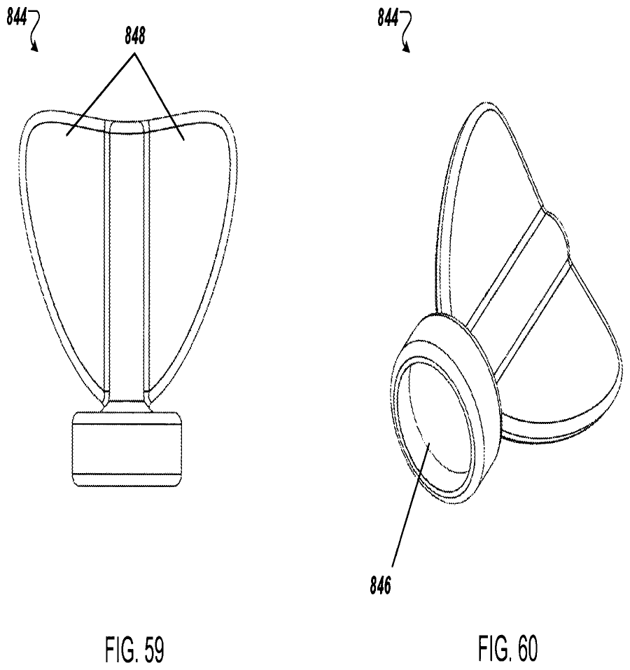

FIG. 59 shows a front view and FIG. 60 shows a perspective view of an alternative implementation of the handle 804, shown as handle 844. Handle 844 includes a space 846 for the driver magnet 802 and two wing grips 848 for turning the handle 844 and the driver magnet 802 about the main axis 830.

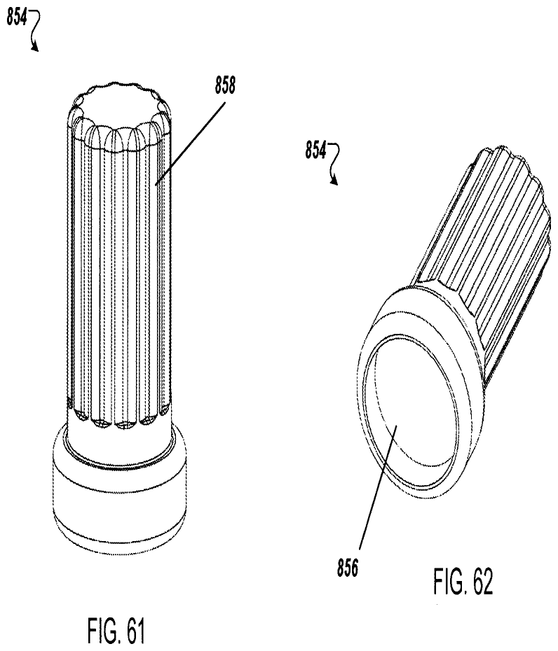

FIG. 61 shows a front view and FIG. 62 shows a perspective view of an alternative implementation of the handle 804, shown as handle 854. Handle 854 includes a space 856 for the driver magnet 802 and gripping cylinder surface 858 for turning the handle 854 and the driver magnet 802 about the main axis 830.

Example Driving System #2

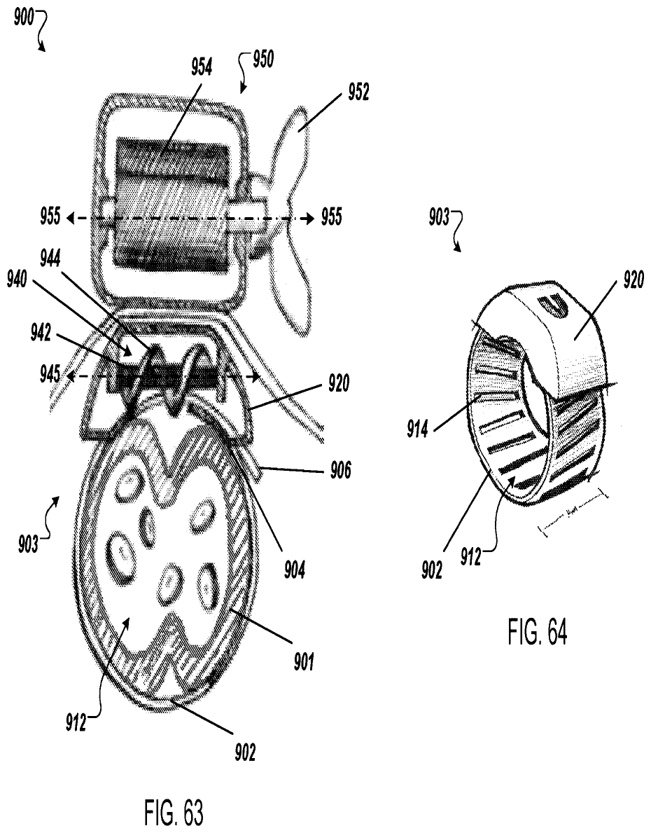

FIG. 63 shows a system 900 for driving an arteriovenous fistula banding device 903. System 900 is similar in structure and function as the above-described implementations, (e.g., device 100 and external driver 800) with a few differences described in this section. FIG. 63 provides a sketch of a cross section of the system 900 including the arteriovenous fistula 901. An arteriovenous fistula banding device 903 is disposed adjacent to the arteriovenous fistula 901 with a band 902 defining a central channel 912. The band 902 includes a first end 904 coupled to a housing 920 and a second end 906 wrapping around the central channel 912 and back through the housing 920. FIG. 64 shows a perspective view of the arteriovenous fistula banding device 903. As shown, the band 902 includes a plurality of slots 914 extending through the band 902 from the inner band surface 908 to the outer band surface 910.

The housing 920 of the arteriovenous fistula banding device 903 includes a magnetic screw 940. In contrast to the screw 140 of device 100, the magnetic screw 940 includes a central magnetic core 942 with at least one thread 944 wrapped around it. The magnetic screw 940 is configured to rotate about a screw axis 945. Similar to the device 100, the at least one thread 944 is configured to abut and engage with the magnetic screw 940 to extend or retract the second end 906 of the band 902, which expands or contracts the diameter of the central channel 912. However, in contrast to the teeth of device 100, the at least one thread 944 of the arteriovenous fistula banding device 903 extend through the plurality of slots 914 in the band 902.

The external driver 950 is placed outside of the skin adjacent to the arteriovenous fistula banding device 903. The external driver 950 includes a handle 952 and an external magnet 954. However, while the external driver 800 of the example driving system #1 includes diametrical disc magnets with aligned axes, the external magnet 954 of example driving system #2 includes an external axis 955 which is parallel to and separated apart from the screw axis 945. Thus, by rotating the handle 952 and the external magnet 954 about the external axis 955, the magnetic screw 940 is driven to rotate about the screw axis 945. The rotation of the magnetic screw 940 moves the band 902 to increase or decrease the diameter of the central channel 912.

Example Driving System #3

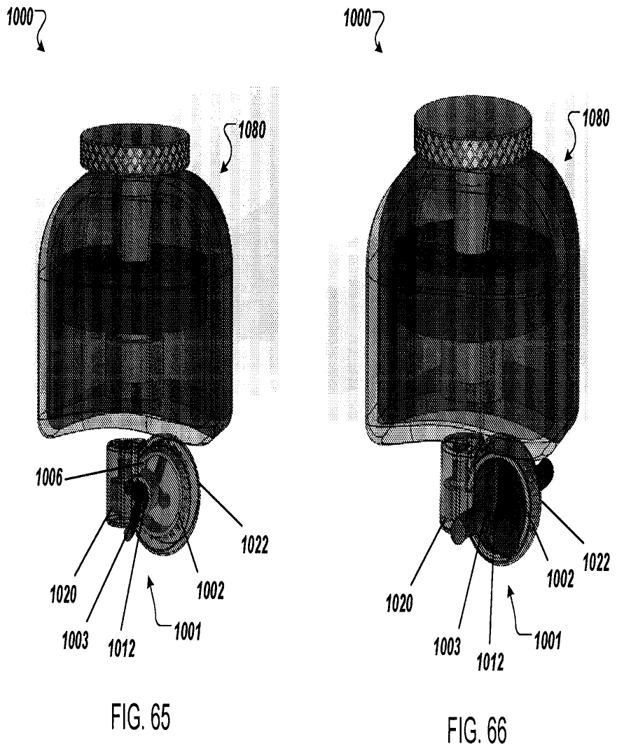

FIGS. 65 and 66 show a system 1000 for driving an arteriovenous fistula banding device 1001. The system 1000 includes an external driver 1080 substantially similar to the external driver 800 of FIGS. 49-58.

The device 1001 is similar to device 100 of FIGS. 3-9. For example, device 1001 includes a band 1002 extending from a housing 1020 and wrapping around an arteriovenous fistula 1003 to define a central channel 1012. However, the housing 1020 of the device 1001 further includes an outer band housing 1022 which circumferentially envelops the band 1002 and the central channel 1012. The second end 1006 of the band 1002 is contained within the outer band housing 1022 regardless of the diameter of the central channel 1012. That is, whether the central channel 1012 has a smaller diameter with the “tail end” of the band 1002 free floating (e.g., the contracted configuration) or a larger diameter (e.g., the expended configuration), the outer band housing 1022 contains the band 1002.

For example, FIG. 65 shows the small diameter contracted configuration where the arteriovenous fistula 1003 is compressed. The tail end or second end 1006 of the band 1002 is not coupled to the directly to the housing 1020, but it is contained within the outer band housing 1022. In FIG. 66, the larger diameter expanded configuration allows the arteriovenous fistula 1003 to expand, but the band 1002 is still contained within the outer band housing 1022. Thus, system 1000 prevents excess movement of the band 1002 within the body of a patient which will reduce complications. In some implementations, the outer band housing 1022 includes an inner track engageable with a portion of the band such that the band follows the track as it extends or retracts.

Experimental Device Testing

Tests were performed to ensure proper operation of performance of the devices and systems described herein. For example, device 100 and external driver 800 were manufactured and tested to ensure that the band 102 would expand or contract in response to rotation of the handle 804. One point of testing was the distance between the magnet 150 and the driver magnet 802 to ensure that rotation of the driver magnet 802 would adequately rotate the magnet 150. Such a distance may be the distance between an implanted device 100 and an external driver 800 disposed on a patient's skin adjacent to the device 100.

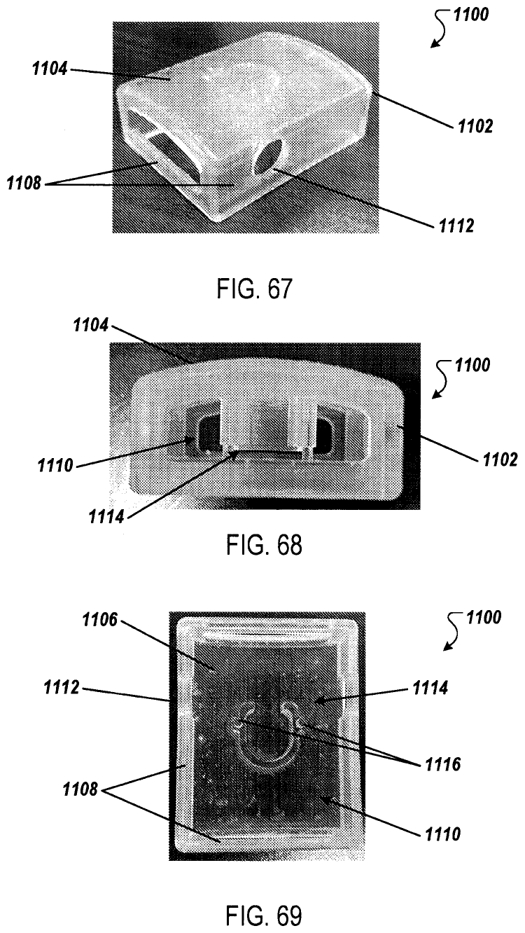

A testing device 1100 was manufactured, as shown in FIGS. 67-69. FIG. 67 shows a perspective view image, FIG. 68 shows a front view image, and FIG. 69 shows a bottom view image of the testing device 1100. The testing device 1100 includes a test housing 1102 having a top surface 1104, a bottom surface 1106, side walls 1108, and an inner cavity 1110 defined by the bottom surface 1106 and the side walls 1108. Each of the side walls 1108 define a port 1112 through which a tube or other device may be placed to simulate an arteriovenous fistula.

The bottom surface 1106 of the testing device 1100 includes a connection rig 1114 including two posts 1116 configured to engage with the suture holes 134 of the device 100 to hold the device 100 in place. The device 100 was placed on the connection rig 1114 via the posts 1116 such that second side 124 of the housing 120 is disposed adjacent to the bottom surface 1106 of the testing device 1100. Thus, the device 100 was “implanted” in the testing device 1100, simulating implanting in a patient.

The external driver 800 was then placed on the top surface 1104 of the testing device 1100. The main axis 830 of the handle 804 and the driver magnet 802 were aligned with the screw axis 155 of the magnet 150 and screw 140 naturally due to the attraction between the magnet 150 and the driver magnet 802. The handle 804 was turned, rotating the driver magnet 802 about the main axis 830. The rotating magnetic field resulted in rotation of the magnet 150 and screw 140 about the screw axis 155. Thus, the at least one thread 142 engaged with the plurality of teeth 114 of the band 102, expanding the diameter of the central channel 112. This test verified the operation of the device 100 with the external driver 800.

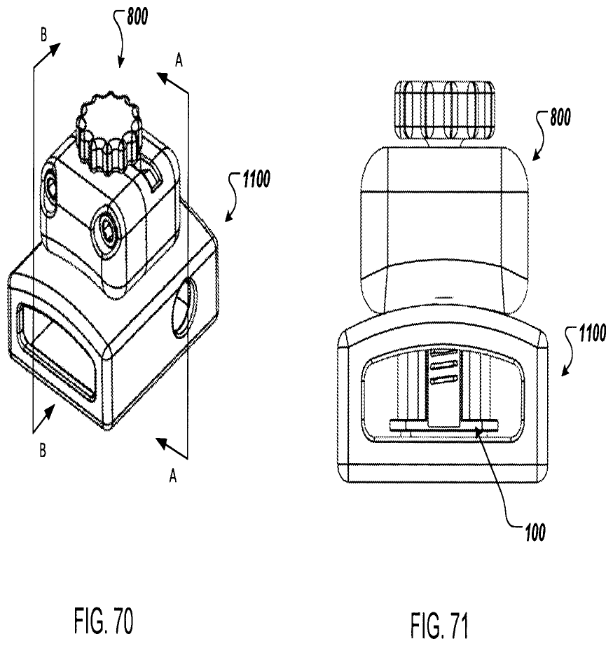

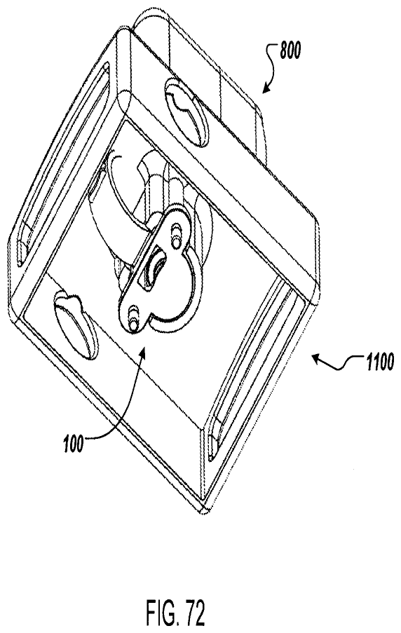

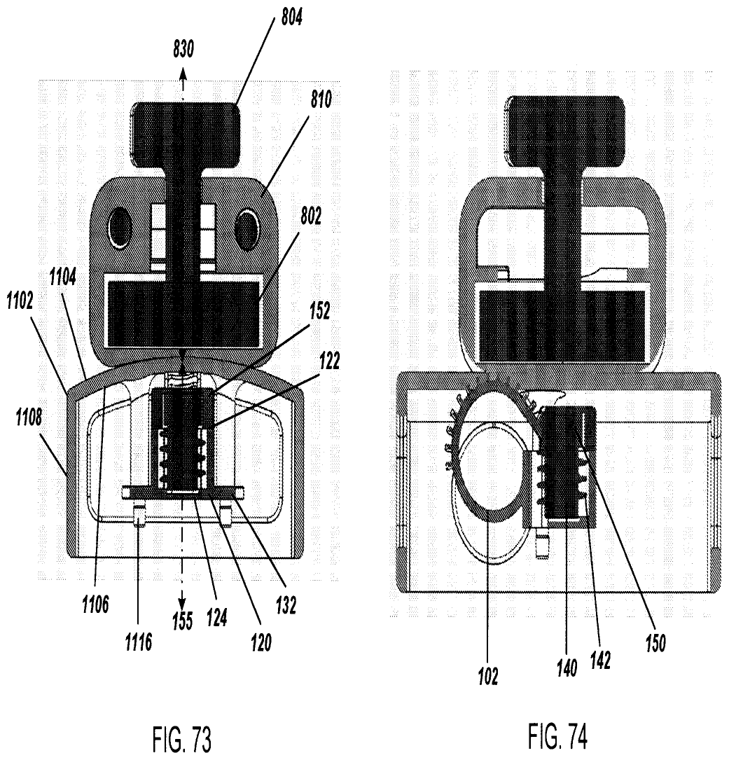

FIG. 70 shows a perspective view of a rendering of the testing device 1100 with the device 100 and the external driver 800 installed thereon for testing. FIG. 71 shows a front view and FIG. 72 shows a bottom perspective view of the same. FIG. 73 shows a cross section of the testing device 1100 across the section AA shown in FIG. 70. FIG. 74 shows a cross section of the testing device 1100 across the section BB shown in FIG. 70. Each of FIGS. 73 and 74 show details on the structure of the testing device 1100 and provide an example understanding of the tests performed. For example, while the band 102 in FIG. 74 appears to interfere with the bottom surface 1106 of the testing device 1100, it is actually disposed underneath the bottom surface 1106 and free to expand or contract within the inner cavity 1110.

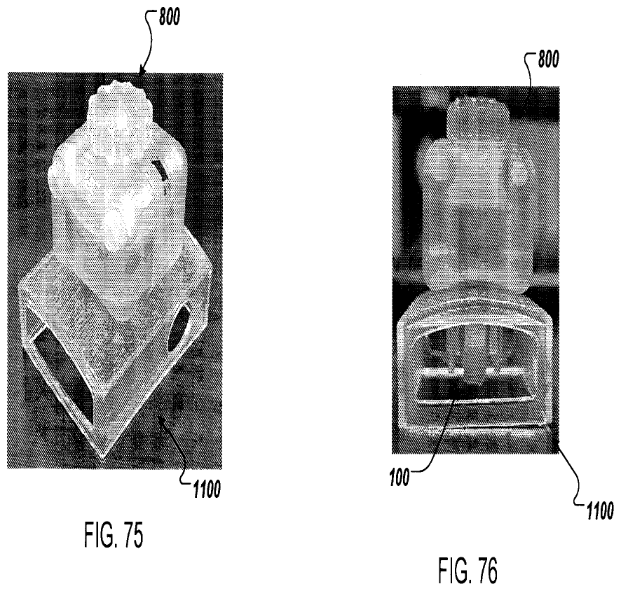

FIGS. 75 and 76 show images of the manufactured prototypes of the testing device 1100 with the external driver 800 and device 100 installed thereon for testing.

Example Method

Described herein are methods for controlling blood flow through an arteriovenous fistula. In one implementation, methods are disclosed for controlling and/or operating an arteriovenous fistula banding device (e.g., device 100) and a corresponding system including an external driver (e.g., external driver 800).

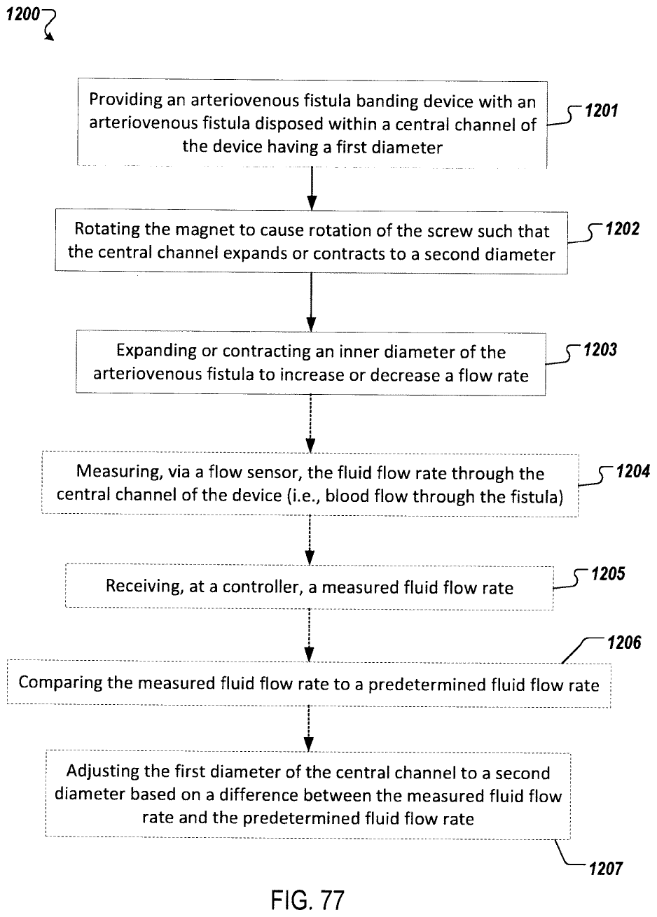

For example, FIG. 77 depicts a flowchart which describes a method 1200 of controlling blood flow through an arteriovenous fistula. The method 1200 includes providing, at step 1201, an arteriovenous fistula device (e.g., the device 100 shown and described in FIGS. 3-9). The device 100 includes a band 102 extending circumferentially to define a central channel 112 having a first diameter, a screw 140 having at least one thread 142 disposed adjacent to or abutting the band 102, and a magnet 150 coupled to the screw 140. An arteriovenous fistula is disposed within the central channel 112.

Next, at step 1202, the method 1200 includes rotating the magnet 150 to cause rotation of the screw 140 such that the at least one thread 142 of the screw 140 contacts a portion of the band 102 to expand or contract the central channel 112 to a second diameter. Such rotation may be accomplished, for example, via an external driver magnet. The external driver magnet may be the driver magnet 802 of the external driver 800 which is disposed outside a patient's body and adjacent to the arteriovenous fistula banding device 100.

The method 1200 then includes, at step 1203, expanding or contracting an inner diameter of the arteriovenous fistula such that a fluid flow rate (e.g., blood flow rate) through the arteriovenous fistula is increased or decreased. Thus, the method 1200 can be implemented to controllably change the blood flow through a patient's arteriovenous fistula. For example, once a device 100 is implanted, the diameter of the fistula may be reduced during normal operation (e.g., a regular day). Then, when the patient receives hemodialysis treatment, the diameter of the fistula is expanded so that the flow rate is increased for the duration of the dialysis treatment. Once dialysis is complete, the fistula is returned to the normal flow rate operation.

Optionally, method 1200 further includes, at step 1204, measuring the fluid flow rate through the central channel 112 via a flow sensor disposed adjacent to the central channel 112 (e.g., the flow sensor 360 in FIG. 20).

Optionally, method 1200 further includes, at step 1205, receiving a measured flow rate at a controller (e.g., the controller 370 of FIG. 20). Optionally, method 1200 further includes, at step 1206, comparing the measured flow rate to a predetermined fluid flow rate (e.g., a desired flow rate of blood through the fistula during dialysis). Optionally, method 1200 further includes, at step 1207, adjusting the diameter of the central channel 112 of the device 100 to a second diameter (e.g., smaller or larger than the first diameter) based on a difference between the measured fluid flow rate and the predetermined fluid flow rate.

Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

1. An arteriovenous fistula banding device, the device comprising:
a band comprising a first end and a second end, the band extending circumferentially around a central axis to define a central channel having a first diameter;
a housing coupled to the first end of the band and defining a housing slot to accept the second end of the band, wherein the housing is disposed outside of the central channel;
a screw disposed within the housing and comprising at least one thread disposed adjacent to or abutting the band; and
an actuator coupled to the screw,
wherein the actuator causes rotation of the screw which causes the second end of the band to move away from or towards the first end of the band such that the first diameter of the central channel changes.

2. The device of claim 1, wherein the actuator is a magnet rotatably disposed within the housing.

3. The device of claim 1, wherein the central channel is sized to surround an arteriovenous fistula, and wherein rotation of the screw to change the first diameter of the central channel changes an inner diameter of the arteriovenous fistula.

4. The device of claim 1, wherein the band further comprises a plurality of teeth protruding from an outer side of the band, wherein the plurality of teeth is engageable with the at least one thread of the screw.

5. The device of claim 1, wherein the band further comprises a plurality of slots extending through the band from an outer side to an inner side, wherein the plurality of slots is engageable with the at least one thread of the screw.

6. The device of claim 1, further comprising a flow sensor disposed adjacent to the central channel, the flow sensor configured to measure fluid flow through the central channel.

7. The device of claim 1, wherein the first diameter of the central channel is adjustable from 3 mm to 15 mm.

8. The device of claim 1, wherein a width of the band as measured in a direction parallel to the central axis is in a range from 5 mm to 10 mm.

9. The device of claim 1, wherein the device comprises a flexible resin and wherein the device is 3D printed in a curved configuration such that the band is biased towards the curved configuration.

10. The device of claim 1, wherein the housing further comprises a magnet cover piece separably couplable to the housing to cover the magnet.

11. The device of claim 1, further comprising a suture protrusion extending out from the housing in a direction transverse to the band, wherein the suture protrusion is disposed outside of the central channel.

12. The device of claim 1, wherein the housing further comprises an outer band housing enveloping the band and the central channel wherein the second end of the band is contained within the outer band housing regardless of the first diameter of the central channel.

13. The device of claim 12, wherein the outer band housing comprises a track engageable with a portion of the band such that the band follows the track as it extends or retracts to expand or contract the central channel.

14. The device of claim 1, wherein the second end of the band further comprises a mechanical stop to define a maximum diameter of the central channel.

15. The device of claim 4, wherein the plurality of teeth extend only from a distal portion of the band such that a proximal portion of the band defines a minimum diameter of the central channel.

16. The device of claim 1, wherein the actuator is a motor with a shaft coupled to the screw.

17. The device of claim 1, further comprising:
a control system comprising:
a flow sensor disposed adjacent to the central channel, the flow sensor configured to measure fluid flow through the central channel; and
a controller configured to receive a measured fluid flow, compare the measured fluid flow to a predetermined fluid flow, and drive the actuator to adjust the first diameter of the central channel based on the predetermined fluid flow.

18. A system comprising:
an arteriovenous fistula banding device, the device comprising:
a band comprising a first end and a second end, the band extending circumferentially around a central axis to define a central channel having a first diameter;
a housing coupled to the first end of the band and defining a housing slot to accept the second end of the band, wherein the housing is disposed outside of the central channel;
a screw disposed within the housing and comprising at least one thread disposed adjacent to or abutting the band; and
a magnet rotatably disposed within the housing coupled to the screw,
wherein rotation of the screw causes the second end of the band to move away from or towards the first end of the band such that the first diameter of the central channel changes; and
an external driver comprising:
a driver magnet; and
a handle coupled to the driver magnet,
wherein rotating the handle of the external driver rotates the driver magnet about a turning axis and causes the magnet of the arteriovenous fistula banding device to rotate.

19-23. (canceled)

24. A method of controlling blood flow through an arteriovenous fistula, the method comprising:
providing an arteriovenous fistula banding device comprising a band extending circumferentially to define a central channel having a first diameter, a screw having at least one thread disposed adjacent to or abutting the band, and a magnet coupled to the screw, wherein the arteriovenous fistula is disposed within the central channel;
rotating the magnet to cause rotation of the screw such that the at least one thread of the screw contacts a portion of the band to expand or contract the central channel to a second diameter; and
expanding or contracting an inner diameter of the arteriovenous fistula such that a fluid flow rate through the arteriovenous fistula is increased or decreased.

25-28. (canceled)
